(19) United States
(12) Patent Application Publication
(10) Pub. No.: US 2022/0323446 A1
(43) Pub. Date: Oct. 13, 2022

(54) SOTORASIB DOSING REGIMEN

(71) Applicant: Amgen Inc., Thousand Oaks, CA (US)

(72) Inventors: Gregory FRIBERG, Westlake Village, CA (US); Haby HENARY, Moorpark, CA (US); Brett E. HOUK, Newbury Park, CA (US); Omar A. MATHER, Thousand Oaks, CA (US); Gataree NGARMCHAMNANRITH, Los Angeles, CA (US); Sandeep DUTTA, Newbury Park, CA (US)

(21) Appl. No.: 17/696,758

(22) Filed: Mar. 16, 2022

Related U.S. Application Data
(60) Provisional application No. 63/162,273, filed on Mar. 17, 2021.
(60) Provisional application No. 63/185,054, filed on May 6, 2021.
(60) Provisional application No. 63/190,096, filed on May 18, 2021.

(51) Int. Cl. A61K 31/519; A61K 31/573; A61K 33/00; A61K 33/10; A61K 33/08; A61K 31/4439; A61K 31/426; A61K 31/341; A61K 31/4164; A61K 31/4453; A61K 31/4545; A61K 31/675; A61K 31/513; A61K 31/4166; A61K 31/496; A61K 31/435; A61K 31/495; A61K 31/536; A61K 31/517; A61K 31/439; A61K 31/7048; A61K 31/445; A61K 31/506; A61K 31/4985; A61K 31/58; A61K 31/40; A61K 31/522; A61K 31/5513; A61K 31/167; A61K 31/635; A61K 31/337; A61P 35/00; A61K 9/00

(52) U.S. Cl. CPC A61K 31/519; A61K 31/573; A61K 33/00; A61K 33/10; A61K 33/08; A61K 31/4439; A61K 31/426; A61K 31/341; A61K 31/4164; A61K 31/4453; A61K 31/4545; A61K 31/675; A61K 31/513; A61K 31/4166; A61K 31/496; A61K 31/435; A61K 31/495; A61K 31/536; A61K 31/517; A61K 31/439; A61K 31/7048; A61K 31/445; A61K 31/506; A61K 31/4985; A61K 31/58; A61K 31/40; A61K 31/522; A61K 31/5513; A61K 31/167; A61K 31/635; A61K 31/337; A61P 35/00; A61K 9/0053

(57) ABSTRACT

Provided herein are methods of treating cancer in a patient comprising administering a total daily dose of 240 mg sotorasib to the patient, wherein the cancer is a KRAS G12C mutated cancer. Also provided herein are methods of treating KRAS G12C mutated cancer in a patient comprising administering a total daily dose of 960 mg sotorasib to the patient, and reducing the total daily dose of sotorasib to 480 mg in a patient experiencing an adverse event to the 960 mg dose of sotorasib.

### CROSS-REFERENCE TO RELATED APPLICATIONS

This application claims the benefit of U.S. Provisional Application No. 63/162,273, filed Mar. 17, 2021, U.S. Provisional Application No. 63/185,054, filed May 6, 2021, and U.S. Provisional Application No. 63/190,096, filed May 18, 2021, all of which are incorporated by reference herein in their entireties.

### BACKGROUND

The rat sarcoma (RAS) proto-oncogene has been identified as an oncogenic driver of tumorigenesis in cancers, such as non-small cell lung cancer (NSCLC) and colorectal cancer (CRC). The RAS family consists of 3 closely related genes that express guanosine triphosphate (GTP)-ases responsible for regulating cellular proliferation and survival. The RAS proteins, Kirsten rat sarcoma viral oncogene homolog (KRAS), Harvey rat sarcoma viral oncogene homolog (HRAS), and neuroblastoma RAS viral oncogene homolog (NRAS) can be mutationally activated at codons 12, 13, or 61, leading to human cancers. Different tumor types are associated with mutations in certain isoforms of RAS, with KRAS being the most frequently mutated isoform in most cancers. While the role of KRAS mutations in human cancers has been known for decades, no anti-cancer therapies specifically targeting KRAS mutations have been successfully developed, until recently, largely because the protein had been considered intractable for inhibition by small molecules.

### SUMMARY

Provided herein are methods of treating cancer in a patient comprising administering a total daily dose of 240 mg sotorasib to the patient, wherein the cancer is a KRAS G12C mutated cancer.

Also provided herein are methods of treating cancer in a patient comprising administering an initial total daily dose of 960 mg sotorasib to the patient, and administering a reduced total daily dose of sotorasib to 480 mg when the patient experiences an adverse event to the initial total daily dose, wherein the cancer is a KRAS G12C mutated cancer. In some embodiments, the methods further comprise administering a second reduced total daily dose of sotorasib of 240 mg when the patient experiences an adverse event to the reduced total daily dose.

In various embodiments, the sotorasib is administered once per day. In various embodiments, the sotorasib is administered orally. In various embodiments, the patient is administered sotorasib for at least one month. In various embodiments, the patient is administered sotorasib for at least three months. In various embodiments, the patient is administered sotorasib for at least six months.

In various embodiments, the cancer is a solid tumor. In various embodiments, the cancer is non-small cell lung cancer, and in some cases, is metastatic or locally advanced and unresectable. In various embodiments, the cancer is colorectal cancer. In various embodiments, the cancer is pancreatic cancer. In various embodiments, the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma.

In various embodiments, the patient, prior to start of sotorasib therapy, had undergone at least one other systemic cancer therapy. In various embodiments, the patient had undergone at least two other systemic cancer therapies. In various embodiments, at least one systemic cancer therapy is selected from anti-PD1 immunotherapy, anti-PDL1 immunotherapy, and platinum-based chemotherapy. In various embodiments, the patient has previously undergone (i) an anti-PD1 therapy or anti-PDL1 therapy, unless contraindicated, or (ii) a platinum-based chemotherapy, and (iii) a EGFR, ALK or ROS1 targeted therapy if the cancer also exhibited a mutation in EGFR, ALK, or ROS1. In various embodiments, the patient has previously undergone (i) an anti-PD1 therapy or anti-PDL1 therapy, unless contraindicated, and (ii) a platinum-based chemotherapy, and (iii) a EGFR, ALK or ROS1 targeted therapy if the cancer also exhibited a mutation in EGFR, ALK, or ROS1.

In various embodiments, the patient does not have active brain metastases within four weeks of the start of sotorasib therapy. In various embodiments, the patient exhibits an Eastern Cooperative Oncology Group (ECOG) performance status of 0, 1, or 2.

In various embodiments, the patient exhibits at least a stable disease (SD) after 1, 3, or 6 months of sotorasib therapy, as measured by RECIST 1.1 protocol. In various embodiments, the stable disease is neither sufficient shrinkage to qualify for partial response (PR) nor sufficient increase to qualify for progressive disease (PD).

In various embodiments, the patient exhibits at least a partial response (PR) after 1, 3, or 6 months of sotorasib therapy, as measured by RECIST 1.1 protocol. In various embodiments, the partial response is at least a 30% decrease in the sum of diameters of target lesions.

In various embodiments, the patient exhibits a progression free survival (PFS) of at least 3 months. In various embodiments, the patient exhibits a PFS of at least 6 months.

In various embodiments, the cancer exhibits a PDL1 tumor proportion score (TPS) of 1-49%. In various embodiments, the cancer exhibits a PDL1 tumor proportion score (TPS) of less than 1%. In various embodiments, the cancer exhibits a PDL1 tumor proportion score (TPS) of 50-100%. In various embodiments, the cancer further comprises a STK11 mutation. In various embodiments, the cancer further comprises a KEAP1 mutation. In various embodiments, the cancer further comprises a STK11 wild-type. In various embodiments, the cancer further comprises a KEAP1 wild-type.

### BRIEF DESCRIPTION OF DRAWINGS

FIG. 1 shows the mean plasma concentration time profile after once daily oral administration of 180, 360, 720, or 960 mg sotorasib on Day 1, where N indicates number of observations across data points.

FIG. 2 shows the mean plasma concentration time profile after once daily oral administration of 180, 360, 720, or 960 mg sotorasib on Day 8, where N indicates number of observations across data points.

FIG. 3 shows a boxplot of best tumor shrinkage of non-small cell lung cancer patients given sotorasib 180 mg QD, 360 mg QD, 720 mg QD, or 960 mg QD, where n is the number of patients, and the percent change from baseline in sum of diameters only considers tumor assessments prior to and including first assessment where timepoint response is progressive disease.

### DETAILED DESCRIPTION

Provided herein are methods of dosing sotorasib to a patient having a cancer with a KRAS G12C mutation. Sotorasib is a small molecule that irreversibly inhibits the KRAS^{G12C} mutant protein. Sotorasib is also referred to as AMG 510 or 6-fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and has the following structure:

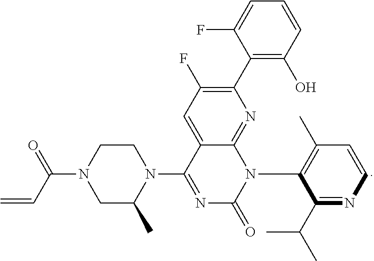

Sotorasib binds to the P2 pocket of KRAS adjacent to the mutant cysteine at position 12 and the nucleotide-binding pocket. The inhibitor contains a thiol reactive portion which covalently modifies the cysteine residue and locks KRAS^{G12C} in an inactive, guanosine diphosphate (GDP) bound conformation. This blocks the interaction of KRAS with effectors such as rapidly accelerated fibrosarcoma (RAF), thereby preventing downstream signaling, including the phosphorylation of extracellular signal regulated kinase (ERK) (Cully and Downward, 2008; Ostrem et al., 2013; Simanshu et al., 2017). Inactivation of KRAS by RNA interference (RNAi) or small molecule inhibition has previously demonstrated an inhibition of cell growth and induction of apoptosis in tumor cell lines and xenografts harboring KRAS mutations (including the KRAS G12C mutation) (Janes et al., 2018; McDonald et al., 2017; Xie et al., 2017; Ostrem and Shokat, 2016; Patricelli et al., 2016). Studies with sotorasib have confirmed these in vitro findings and have likewise demonstrated inhibition of growth and regression of cells and tumors harboring KRAS G12C mutations (Canon et al., 2019).

In various embodiments of the disclosure, the patient is administered a total daily dose of 240 mg sotorasib. In some embodiments, the sotorasib is administered once daily. In various embodiments, the sotorasib is administered orally. In various embodiments, the sotorasib is administered with food. In various embodiments, the sotorasib is administered without food.

In various embodiments, the patient is further in need of treatment with an acid-reducing agent. Acid-reducing agents include, but are not limited to a proton pump inhibitor (PPI), a H2 receptor antagonist (H2RA), and a locally acting antacid. In one embodiment, the patient is further in need of treatment with a PPI or a H2RA. Exemplary PPIs include, but are not limited to, esomeprazole, lansoprazole, rabeprazole, and dexlansoprazole. Exemplary PPIs include, but are not limited to, omeprazole, pantoprazole, esomeprazole, lansoprazole, rabeprazole, or dexlansoprazole. Exemplary H2RAs include, but are not limited to, famotidine, ranitidine, cimetidine, nizatidine, roxatidine and lafutidine. Exemplary locally acting antacids include, but are not limited to, sodium bicarbonate, calcium carbonate, aluminum hydroxide, and magnesium hydroxide. In some embodiments, the patient is not administered a proton pump inhibitor or a H2 receptor antagonist in combination with sotorasib. In some embodiments, provided the patient is further in need of treatment with an acid-reducing agent, sotorasib is administered about 4 hours before or about 10 hours after a locally acting antacid.

In various embodiments, the patient is in further need of treatment with a CYP3A4 inducer. In some embodiments, the patient is not administered a CYP3A4 inducer in combination with sotorasib. Exemplary CYP3A4 inducers include, but are not limited to, barbiturates, brigatinib, carbamazepine, clobazam, dabrafenib, efavirenz, elagolix, enzalutamide, eslicarbazepine, glucocorticoids, letermovir, lorlatinib, modafinil, nevirapine, oritavancin, oxcarbazepine, perampanel, phenobarbital, phenytoin, pioglitazone, rifabutin, rifampin, telotristat, and troglitazone. See, e.g., Flockhart DA, Drug Interactions: Cytochrome P450 Drug Interaction Table. Indiana University School of Medicine (2007), www.drug-interactions.medicine.iu.edu, accessed May 2021. In some embodiments, the patient is not administered a strong CYP3A4 inducer in combination with sotorasib. Exemplary strong CYP3A4 inducers include, but are not limited to, phenytoin and rifampin. See, e.g., www.fda.gov/drugs/drug-interactions-labeling/drug-development-and-drug-interactions-table-substrates-inhibitors-and-inducers, accessed May 2021.

In various embodiments, the patient is in further need of treatment with a CYP3A4 substrate. In some embodiments, the patient is not administered a CYP3A4 substrate in combination with sotorasib. Exemplary CYP3A4 substrates include, but are not limited to, abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, gleevec, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus(fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, and ziprasidone. See, e.g., Flockhart DA, Drug Interactions: Cytochrome P450 Drug Interaction Table. Indiana University School of Medicine (2007), https://drug-interactions.medicine.iu.edu, accessed May 2021.

In some embodiments, the patient is not administered a CYP3A4 substrate in combination with sotorasib, wherein the CYP3A4 substrate is a CYP3A4 substrate with a narrow therapeutic index. Exemplary CYP3A4 substrates with a narrow therapeutic index include, but are not limited to, alfentanil, fentanyl, cyclosporine, pimozide, dihydroergotamine, quinidine, ergotamine, sirolimus, everolimus, and tacrolimus.

In various embodiments, the patient is in further need of treatment with a P-glycoprotein (P-gp) substrate. In some embodiments, the patient is not administered a P-gp substrate in combination with sotorasib. Exemplary P-gp substrates include, but are not limited to dabigatran etexilate, digoxin, and fexofenadine. See, e.g., www.fda.gov/drugs/drug-interactions-labeling/drug-development-and-drug-interactions-table-substrates-inhibitors-and-inducers, accessed May 2021. In some embodiments, the patient is not administered a P-gp substrate in combination with sotorasib, wherein the P-gp substrate is a P-gp substrate with a narrow therapeutic index. Exemplary P-gp substrates with a narrow therapeutic index include, but are not limited to, digoxin, everolimus, cyclosporine, sirolimus, tacrolimus, and vincristine. P-gp subtrates with a narrow therapeutic index are compounds for which minimal concentration changes may lead to serious toxicities.

In various embodiments, the patient has a cancer that was determined to have one or more cells expressing the KRAS^{G12C} mutant protein prior to administration of sotorasib as disclosed herein. Determination of KRAS^{G12C} mutant protein can be assessed as described elsewhere in this disclosure.

The patient administered 240 mg sotorasib in the methods disclosed herein can have been previously treated with a systemic cancer therapy, e.g., at least one—such as one, or two, or three—other systemic cancer therapy. In some embodiments, the patient administered the sotorasib in the methods described herein has not previously been treated with a systemic cancer therapy. In some embodiments, the patient had previously been treated with one other systemic cancer therapy, such that the sotorasib therapy is a second line therapy. In some embodiments, the patient had previously been treated with two other systemic cancer therapy, such that the sotorasib therapy is a third line therapy.

In some embodiments, the prior systemic cancer therapy is a therapy with a KRAS^{G12C} inhibitor. In some embodiments, the prior systemic cancer therapy is not a therapy with a KRAS^{G12C} inhibitor. In certain embodiments, the patient exhibits reduced sensitivity to a therapy with a KRAS^{G12C} inhibitor. In some embodiments, the patient is resistant to a therapy with a KRAS^{G12C} inhibitor. In some embodiments, KRAS^{G12C} inhibitor is sotorasib, adagrasib, GDC-6036, D-1553, JDQ443, LY3537982, B11823911, JAB-21822, RMC-6291, or APG-1842. In certain embodiments the KRAS^{G12C} inhibitor is sotorasib. In certain embodiments, the KRAS^{G12C} inhibitor is adagrasib. In some embodiments, the therapy is monotherapy. In one embodiment, the therapy with a KRAS^{G12C} inhibitor is sotorasib monotherapy. In another embodiment, the therapy with a KRAS^{G12C} inhibitor is monotherapy with adagrasib.

As used herein “sensitivity” refers to the way a cancer reacts to a drug, e.g., sotorasib. In exemplary aspects, “sensitivity” means “responsive to treatment” and the concepts of “sensitivity” and “responsiveness” are positively associated in that a cancer or tumor that is responsive to a drug treatment is said to be sensitive to that drug. “Sensitivity” in exemplary instances is defined according to Pelikan, Edward, Glossary of Terms and Symbols used in Pharmacology (Pharmacology and Experimental Therapeutics Department Glossary at Boston University School of Medicine), as the ability of a population, an individual or a tissue, relative to the abilities of others, to respond in a qualitatively normal fashion to a particular drug dose. The smaller the dose required producing an effect, the more sensitive is the responding system. “Sensitivity” may be measured or described quantitatively in terms of the point of intersection of a dose-effect curve with the axis of abscissal values or a line parallel to it; such a point corresponds to the dose just required to produce a given degree of effect. In analogy to this, the “sensitivity” of a measuring system is defined as the lowest input (smallest dose) required producing a given degree of output (effect). In exemplary aspects, “sensitivity” is opposite to “resistance” and the concept of “resistance” is negatively associated with “sensitivity”. For example, a cancer that is resistant to a drug treatment is either not sensitive nor responsive to that drug or was initially sensitive to the drug and is no longer sensitive upon acquiring resistance; that drug is not or no longer an effective treatment for that tumor or cancer cell.

Prior systemic cancer therapies include, but are not limited to, chemotherapies and immunotherapies. Specific contemplated prior systemic cancer therapies include anti-PD1 therapy, anti-PDL1 therapy, platinum based chemotherapy, and anti-EGFR therapy. Some examples of anti-PD1 therapy and anti-PDL1 therapies include, but are not limited to, pembrolizumab, nivolumab, cemiplimab, tisielizumab, toripalimab, aspartalizumab, dostarlimab, retifanlimab, simtilimab, pidilizumab atezolizumab, avelumab, and durvalumab. In some embodiments, the anti-PD1 therapy or anti-PDL1 therapy is balstilimab, budigalimab, cadonilimab, camrelizumab, cetrelimab, cemiplimab, dostarlimab, ezabenlimab, finotonlimab, nivolumab, penpulimab, pembrolizumab, pucotenlimab, retifanlimab, rulonilimab, sasanlimab, serplulimab, sintilimab, spartalizumab, tebotelimab, tislelizumab, toripalimab, zeluvalimab (AMG 404), and zimberelimab. In certain embodiments the anti-PD1 therapy or antibody is cemiplimab, dostarlimab, pembrolizumab, or nivolumab. Some examples of anti-PDL1 therapies or antibodies include, but are not limited to, adebrelimab, atezolizumab, avelumab, cosibelimab, durvalumab, envafolimab, erfonrilimab, garivulimab, lodapolimab, opucolimab, sugemalimab, socazolimab, and tagitanlimab. In some embodiments the anti-PDL1 therapy or antibody is atezolizumab, avelumab, or durvalumab. Some examples of platinum based chemotherapies include, but are not limited to, carboplatin, oxaliplatin, cisplatin, nedaplatin, satraplatin, lobaplatin, triplatin tetranitrate, picoplatin, ProLindac™ (AP5346), and aroplatin. Some examples of anti-EGFR therapy include, but are not limited to, cetuximab and panitumumab.

In some embodiments, the patient has previously been administered a systemic cancer therapy that is a targeted therapy if the cancer was identified to have an actionable oncogenic driver mutation in the epidermal growth factor receptor gene (EGFR), anaplastic lymphoma kinase gene (ALK), and/or ROS proto-oncogene 1 (ROS1). Targeted therapies for EGFR mutations include, but are not limited to, cetuximab, panitumumab, erlotinib, gefitinib, and afatinib. Targeted therapies for ALK mutations include, but are not limited to, crizotinib, entrectinib, lorlatinib, repotrectinib, brigatinib, alkotinib, alectinib, ensartinib, and ceritinib. Targeted therapies for ROS1 mutations include, but are not limited to, crizotinib, entrecetinib, ensartinib, alkotinib, brigatinib, taletrectinib, cabozantinib, repotrectinib, lorlatinib, and ceritinib.

In various embodiments, the patient does not exhibit active brain metastases. In some embodiments, the patient does not exhibit brain metastases within 4 weeks of the start of sotorasib therapy as disclosed herein.

In various embodiments, the patient exhibits an Eastern Cooperative Oncology Group (ECOG) performance status of 0, 1, or 2 (see, e.g., Zubrod et al., 1960). Status 0 indicates fully active and able to carry on all pre-disease performance without restriction. Status 1 indicates restricted in physically strenuous activity but ambulatory and able to carry out work of a light or sedentary nature. Status 2 indicates ambulatory and capable of all selfcare but unable to carry out any work activities; up and about more than 50% of waking hours. Status 3 indicates capable of only limited selfcare, confined to bed or chair more than 50% of waking hours. Status 4 indicates completely disabled, cannot carry on any selfcare and totally confined to bed or chair. Status 5 indicates death.

Dose Modification Scheme

Also provided herein are methods of treating cancer in a patient comprising administering an initial total daily dose of 960 mg sotorasib to the patient, and administering a reduced total daily dose of sotorasib of 480 mg when the patient experiences an adverse event to the initial total daily dose, wherein the cancer is a KRAS G12C mutated cancer. In some embodiments, the methods further comprise administering a second reduced total daily dose of sotorasib of 240 mg when the patient experiences an adverse event to the reduced total daily dose.

The term “adverse event or (AE)” as used herein refers to any unfavorable and unintended sign (including an abnormal laboratory finding), symptom, or disease temporally associated with the use of a medical treatment or procedure that may be considered related to the medical treatment or procedure.

In some embodiments, the adverse event is hepatotoxicity (e.g., elevation of liver enzymes), diarrhea, and/or nausea/vomiting. In some embodiments, the adverse event is hepatotoxicity (e.g., elevation of liver enzymes), interstitial lung disease/pneumonitis, diarrhea, and/or nausea/vomiting.

Hepatotoxicity

In some embodiments, the adverse event is hepatotoxicity. The term “hepatotoxicity” as used herein refers to a patient having abnormal laboratory values of liver biomarkers (e.g., alkaline phosphatase (ALP), aspartate amino transferase (AST), alanine aminotransferase (ALT), and/or total bilirubin (TBL)), when the patient had baseline levels of the liver biomarker(s) prior to sotorasib administration that were not abnormal laboratory values or were lower than those measured after administration of sotorasib.

Alanine transaminase (ALT), also called serum glutamic pyruvate transaminase (SGPT) or alanine aminotransferase (ALAT), catalyzes the transfer of an amino group from alanine to α-ketoglutarate to produce pyruvate and glutamate. When the liver is damaged, levels of ALT in the blood can rise due to the leaking of ALT into the blood from damaged or necrosed hepatocytes.

Aspartate transaminase (AST) also called serum glutamic oxaloacetic transaminase (SGOT or GOT) or aspartate aminotransferase (ASAT), catalyzes the transfer of an amino group from aspartate to α-ketoglutarate to produce oxaloacetate and glutamate. AST can increase in response to liver damage. Elevated AST also can result from damage to other sources, including red blood cells, cardiac muscle, skeletal muscle, kidney tissue, and brain tissue. The ratio of AST to ALT can be used as a biomarker of liver damage.

Bilirubin is a catabolite of heme that is cleared from the body by the liver. Conjugation of bilirubin to glucuronic acid by hepatocytes produces direct bilirubin, a water-soluble product that is readily cleared from the body. Indirect bilirubin is unconjugated, and the sum of direct and indirect bilirubin constitutes total bilirubin. Elevated total bilirubin can be indicative of liver impairment.

Alkaline phosphatase (ALP) hydrolyzes phosphate groups from various molecules and is present in the cells lining the biliary ducts of the liver. ALP levels in plasma can rise in response to liver damage and are higher in growing children and elderly patients with Paget's disease. However, elevated ALP levels usually reflect biliary tree disease.

In some embodiments, the patient is not suffering from a disorder that results in elevated liver biomarkers. Disorders associated with elevated liver biomarkers (such as AST/ALT and/or TBL values) include, but are not limited to, hepatobiliary tract disease; viral hepatitis (e.g., hepatitis A/B/C/D/E, Epstein-Barr Virus, cytomegalovirus, herpes simplex virus, varicella, toxoplasmosis, and parvovirus); right sided heart failure, hypotension or any cause of hypoxia to the liver causing ischemia; exposure to hepatotoxic agents/drugs or hepatotoxins, including herbal and dietary supplements, plants and mushrooms; heritable disorders causing impaired glucuronidation (e.g., Gilbert's syndrome, Crigler-Najjar syndrome) and drugs that inhibit bilirubin glucuronidation (e.g., indinavir, atazanavir); alpha-one antitrypsin deficiency; alcoholic hepatitis; autoimmune hepatitis; Wilson's disease and hemochromatosis; nonalcoholic fatty liver disease including steatohepatitis; and/or non-hepatic causes (e.g., rhabdomyolysis, hemolysis).

Prior to receiving sotorasib, the baseline liver function of the patient can be assessed by various means known in the art, such as blood chemistry tests measuring biomarkers of liver function. In some embodiments, the methods described herein comprise monitoring liver biomarkers in the patient and withholding sotorasib administration in patients having >Grade 2 abnormal liver function, as assessed by levels of AST and/or ALT. In such embodiments, sotorasib administration is paused until the AST and/or ALT levels in the patient improve(s) to Grade 1 or better (baseline).

Adverse effect Grades for abnormal liver function are defined herein by the modified Common Toxicity Criteria (CTC) provided in Table 1. See the National Cancer Institute Common Terminology Criteria for Adverse Events v5.0 (NCI CTCAE) published Nov. 27, 2017 by the National Cancer Institute, incorporated herein by reference in its entirety.

TABLE 1
Common Toxicity Criteria
TOXICITY GRADES

| TOXICITY | 0 | 1 | 2 | 3 | 4 |
|---|---|---|---|---|---|
| ALT | WNL | >ULN-3.0 × ULN, | >3-5 × ULN, if | >5-20 × ULN, if | >20 × ULN if |
| | | if baseline was | baseline was | baseline was | baseline was |
| | | normal; 1.5- | normal, >3.0- | normal; >5.0- | normal; >20 × |
| | | 3.0 × baseline | 5.0 × baseline | 20.0 × baseline | baseline if |
| | | is baseline | if baseline | if baseline | baseline |
| | | was abnormal | was abnormal | was abnormal | was abnormal |
| AST | WNL | >ULN-3.0 × ULN | >3-5 × ULN if | >5-20 × ULN if | >20 × ULN if |
| | | if baseline was | baseline was | baseline was | baseline was |
| | | normal; 1.5- | normal, >3.0- | normal; >5.0- | normal; >20 × |
| | | 3.0 × baseline | 5.0 × baseline | 20.0 × baseline | baseline if |
| | | is baseline | id baseline | if baseline | baseline |
| | | was abnormal | was abnormal | was abnormal | was abnormal |
| Bilirubin | WNL | >ULN-1.5 × ULN if | >1.5-3 × ULN if | >3-10 × ULN if | >10 × ULN if |
| | | baseline was | baseline was | baseline was | baseline was |
| | | normal; >1.0- | normal; >1.5- | normal; >3.0- | normal; >10.0 × |
| | | 1.5 × baseline | 3.0 × baseline | 10 × baseline | baseline if |
| | | if baseline | if baseline | if baseline | baseline |
| | | was abnormal | was abnormal | was abnormal | was abnormal |
| ALP | WNL | >ULN-2.5 × ULN | >2.5-5.0 × ULN if | >5-20 × ULN if | >20 × ULN if |
| | | if baseline was | baseline was | baseline was | baseline was |
| | | normal; 2.0- | normal, >2.5- | normal; >5.0- | normal; >20 × |
| | | 2.5 × baseline | 5.0 × baseline | 20.0 × baseline | baseline if |
| | | is baseline | if baseline | if baseline | baseline |
| | | was abnormal | was abnormal | was abnormal | was abnormal |

ALP = alkaline phosphatase;
ALT = alanine aminotransferase;
AST = aspartate aminotransferase;
ULN = upper limit of normal;
WNL = within normal limits

Grade 0 levels are characterized by biomarker levels within normal limits (WNL). “Normal” liver function, as used herein, refers to Grade 0 adverse effects. “Abnormal” liver function, as used herein, refers to Grade 1 and above adverse effects.

“Grade 1 liver function abnormalities” include elevations in ALT or AST greater than the ULN and less than or equal to 3-times the ULN if baseline was normal; 1.5-3.0× baseline is baseline was abnormal. Grade 1 liver function abnormalities also include elevations of bilirubin levels greater than the ULN and less than or equal to 1.5-times the ULN if baseline was normal; >1.0-1.5× baseline if baseline was abnormal. Grade 1 liver function abnormalities also include elevations of ALP greater than the ULN and less than or equal to 2.5-times the ULN if baseline was normal; >2.0-2.5× baseline if baseline was abnormal.

“Grade 2 liver function abnormalities” include elevations in ALT or AST greater than 3-times and less than or equal to 5-times the upper limit of normal (ULN) if baseline was normal, >3.0-5.0× baseline if baseline was abnormal. Grade 2 liver function abnormalities also include elevations of bilirubin levels greater than 1.5-times and less than or equal to 3-times the ULN if baseline was normal; >1.5-3.0× baseline if baseline was abnormal. Grade 2 liver function abnormalities also include elevations of ALP greater than 2.5-times and less than or equal to 5-times the ULN if baseline was normal; >2.5-5.0× baseline if baseline was abnormal.

“Grade 3 liver function abnormalities” include elevations in ALT, AST, or ALP greater than 5-times and less than or equal to 20-times the ULN if baseline was normal; >5.0-20.0× baseline if baseline was abnormal. Grade 3 liver function abnormalities also include elevations of bilirubin levels greater than 3-times and less than or equal to 10-times the ULN if baseline was normal; >3.0-10× baseline if baseline was abnormal.

“Grade 4 liver function abnormalities” include elevations in ALT, AST, or ALP greater than 20-times the ULN if baseline was normal; >20× baseline if baseline was abnormal. Grade 4 liver function abnormalities also include elevations of bilirubin levels greater than 10 times the ULN if baseline was normal; >10.0× baseline if baseline was abnormal.

The ULN for various indicators of liver function depends on the assay used, the patient population, and each laboratory's normal range of values for the specified biomarker, but can readily be determined by the skilled practitioner. Exemplary values for normal ranges for a healthy adult population are set forth in Table 2 below. See Cecil Textbook of Medicine, pp. 2317-2341, W.B. Saunders & Co. (1985).

TABLE 2
Upper Limit of Normal (ULN) Values

| | | |
|---|---|---|
| ALT | 8-20 | U/L |
| AST | 8-20 | U/L |
| Bilirubin | 0.2-1 | mg/dL |
| | 3.4-17.1 | μmol/L |
| ALP | 20-70 | U/L |

In any of the methods described herein, the total daily dose of sotorasib is reduced (e.g., from 960 mg to 480 mg, or from 480 mg to 240 mg) when the AST and/or ALT level(s) in the patient is/are elevated, e.g. to a Grade 2 or Grade 3 level, where the baseline AST and/or ALT levels of the patient were below Grade 2 or Grade 3 levels. In some embodiments, the total daily dose of sotorasib is reduced (e.g., from 960 mg to 480 mg, or from 480 mg to 240 mg), when the AST and/or ALT level(s) in the patient is/are elevated is to a Grade 1 level, wherein the baseline AST and/or ALT levels of the patient were below Grade 1 levels.

Alternatively, in any of the methods disclosed herein, the total daily dose of sotorasib is reduced (e.g., from 960 mg to 480 mg, or from 480 mg to 240 mg) when (1) AST and bilirubin levels in the patient are elevated, or (2) when AST or ALP levels in the patient are elevated, or (3) when ALT and bilirubin levels in the patient are elevated, or (4) when ALT and ALP levels in the patient are elevated, or (5) when bilirubin and ALP levels in the patient are elevated, e.g., to a Grade 1, Grade 2, Grade 3 or Grade 4 level, wherein the baseline AST, bilirubin, ALP, and/or ALT levels of the patient were below Grade 1, Grade 2, Grade 3 or Grade 4 levels, respectively. Alternatively, in any of the methods disclosed herein three biomarkers of liver function may be elevated in the patient, e.g., ALT and AST and bilirubin, or ALT and AST and ALP, to a Grade 1, Grade 2, Grade 3 or Grade 4 level, wherein the baseline biomarker levels of the patient were below Grade 1, Grade 2, Grade 3 or Grade 4 levels, respectively.

In some embodiments, the total daily dose of sotorasib is reduced (e.g., from 960 mg to 480 mg, or from 480 mg to 240 mg) when the level of ALT and/or AST is greater than about 3 times compared to the upper limit of normal (ULN). In a related embodiment, the abnormal level of ALT and/or AST is greater than about 3- to about 5-fold increase compared to the upper limit of normal (ULN), i.e. a “Grade 2 abnormality”. In some embodiments, where the patient has an abnormal baseline, the Grade 2 abnormality is an abnormal level of ALT and/or AST greater than about 3-fold to about 5-fold increase compared to baseline. In some embodiments, the abnormal level of ALP is greater than about 2.5- to about 5-fold increase compared to the upper limit of normal (ULN), i.e., a “Grade 2 abnormality”. In some embodiments, where the patient has an abnormal baseline, the Grade 2 abnormality is an abnormal level of ALP greater than about 2.5-fold to about 5-fold increase compared to baseline. In some embodiments, the abnormal level of bilirubin is greater than about 1.5- to about 3-fold increase compared to the upper limit of normal (ULN), i.e., a “Grade 2 abnormality”. In some embodiments, where the patient has an abnormal baseline, the Grade 2 abnormality is an abnormal level of bilirubin greater than about 1.5-fold to about 3-fold increase compared to baseline.

In some embodiments, the total daily dose of sotorasib is reduced (e.g., from 960 mg to 480 mg, or from 480 mg to 240 mg) when the level of ALT and/or AST, is greater than about 5 times compared to the upper limit of normal (ULN). In some embodiments, the total daily dose is reduced when the level of ALT, AST, or ALP is greater than about 5- to about 20-fold increase compared to the upper limit of normal (ULN), i.e. a “Grade 3 abnormality”. In some embodiments, where the patient has an abnormal baseline, the Grade 3 abnormality is an abnormal level of ALT and/or AST greater than about 5-fold to about 20-fold increase compared to baseline. In some embodiments, the abnormal level of ALP is greater than about 5- to about 20-fold increase compared to the upper limit of normal (ULN), i.e., a “Grade 3 abnormality”. In some embodiments, where the patient has an abnormal baseline, the Grade 3 abnormality is an abnormal level of ALP greater than about 5-fold to about 20-fold increase compared to baseline. In some embodiments, the total daily dose is reduced when the level of bilirubin is greater than about 3- to about 10-fold increase compared to the upper limit of normal (ULN), i.e., a “Grade 3 abnormality”. In some embodiments, where the patient has an abnormal baseline, the Grade 3 abnormality is an abnormal level of bilirubin greater than about 3-fold to about 10-fold increase compared to baseline.

In some embodiments, the total daily dose of sotorasib is reduced (e.g., from 960 mg to 480 mg, or from 480 mg to 240 mg) when the level of ALT and/or AST is greater than about 20 times compared to the upper limit of normal (ULN) (i.e., a “Grade 4 abnormality”). n some embodiments, where the patient has an abnormal baseline, the Grade 4 abnormality is an abnormal level of ALT and/or AST greater than about 20-fold increase compared to baseline. In some embodiments, the abnormal level of ALP is greater than about 20-fold increase compared to the upper limit of normal (ULN), i.e., a “Grade 4 abnormality”. In some embodiments, where the patient has an abnormal baseline, the Grade 4 abnormality is an abnormal level of ALP greater than about 20-fold increase compared to baseline. In some embodiments, the total daily dose is reduced when the level of bilirubin is greater than about 10-fold increase compared to the upper limit of normal (ULN), i.e., a “Grade 4 abnormality”. In some embodiments, where the patient has an abnormal baseline, the Grade 4 abnormality is an abnormal level of bilirubin greater than about 10-fold increase compared to baseline.

In some embodiments, the methods described herein further comprise increasing the total dose of sotorasib (e.g., from 240 mg to 480 mg, or from 480 mg to 960 mg) when liver biomarker(s) in the patient has improved to a Grade 1 or better (e.g., baseline).

Nausea/Vomiting

In some embodiments, the adverse event is nausea or vomiting. In some embodiments, the nausea/vomiting is present despite appropriate supportive care (e.g., anti-emetic therapy). “Nausea” as used herein refers to a disorder characterized by a queasy sensation and/or the urge to vomit.

Adverse effect Grades for nausea and vomiting are defined herein by the modified Common Toxicity Criteria (CTC) provided in Table 3. See the National Cancer Institute Common Terminology Criteria for Adverse Events v5.0 (NCI CTCAE) published Nov. 27, 2017 by the National Cancer Institute, incorporated herein by reference in its entirety.

TABLE 3

| | Grade 1 | Grade 2 | Grade 3 | Grade 4 |
|---|---|---|---|---|
| Nausea | Loss of appetite | Oral intake | Inadequate oral | — |
| | without alteration in | decreased without | caloric or fluid | |
| | eating habits | significant weight | intake; tube | |
| | | loss, dehydration or | feeding, TPN, or | |
| | | malnutrition | hospitalization | |
| | | | indicated | |
| Vomiting | Intervention not | Outpatient IV | Tube feeding, TPN, | Life-threatening |
| | indicated | hydration; medical | or hospitalization | consequences |
| | | intervention | indicated | |
| | | indicated | | |

In some embodiments, the methods described herein comprise withholding sotorasib administration in a patient having >Grade 3 nausea until the patient has improved to Grade 1 or baseline. In some embodiments, once the patient has improved to Grade 1 or baseline, the methods comprise administering a reduced total daily dose of sotorasib (e.g., from 960 mg to 480 mg, or from 480 mg to 240 mg) to the patient.

In some embodiments, the methods described herein comprise withholding sotorasib administration in a patient having >Grade 3 vomiting until the vomiting improves to Grade 1 or baseline. In some embodiments, once the patient has improved to Grade 1 or baseline, the methods comprise administering a reduced total daily dose of sotorasib (e.g., from 960 mg to 480 mg, or from 480 mg to 240 mg) to the patient.

In some embodiments, the methods described herein further comprise increasing the total dose of sotorasib (e.g., from 240 mg to 480 mg, or from 480 mg to 960 mg) when the nausea or vomiting in the patient has improved to a Grade 1 or better (e.g., baseline).

Diarrhea

In some embodiments, the adverse event is diarrhea. In some embodiments, the diarrhea is present despite appropriate supportive care (e.g., anti-diarrheal therapy).

Adverse effect Grades for diarrhea are defined herein by the modified Common Toxicity Criteria (CTC) provided in Table 4. See the National Cancer Institute Common Terminology Criteria for Adverse Events v5.0 (NCI CTCAE) published Nov. 27, 2017 by the National Cancer Institute, incorporated herein by reference in its entirety.

TABLE 4

| | Grade 1 | Grade 2 | Grade 3 | Grade 4 |
|---|---|---|---|---|
| Diarrhea | Increase of <4 | Increase of 4-6 | Increase of >7 | Life-threatening |
| | stools per day over | stools per day over | stools per day over | consequences; |
| | baseline; mild | baseline; moderate | baseline; | urgent intervention |
| | increase in ostomy | increase in ostomy | hospitalization | indicated |
| | output compared to | output compared to | indicated; severe | |
| | baseline | baseline; limiting | increase in ostomy | |
| | | instrumental | output compared to | |
| | | activities of daily | baseline; limiting | |
| | | life (ADL) | self care ADL | |

In some embodiments, the methods described herein comprise withholding sotorasib administration in a patient having >Grade 3 diarrhea until the patient has improved to Grade 1 or baseline. In some embodiments, once the patient has improved to Grade 1 or baseline, the methods comprise administering a reduced total daily dose of sotorasib (e.g., from 960 mg to 480 mg, or from 480 mg to 240 mg) to the patient.

In some embodiments, the methods described herein further comprise increasing the total dose of sotorasib (e.g., from 240 mg to 480 mg, or from 480 mg to 960 mg) when diarrhea in the patient has improved to a Grade 1 or better (e.g., baseline).

### Response to Sotorasib Therapy

Response rates or results for patients administered a 240 mg sotorasib total daily dose in the methods disclosed herein can be measured in a number of ways, after the patient has been taking the 240 mg sotorasib therapy for a suitable length of time. In various embodiments, a patient is administered 240 mg total daily dose of sotorasib for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months, e.g., for 1 month, 2 months, 3 months, 4 months, 5 months, 6 months, 7 months, 8 months, 9 months, 10 months, 11 months, 12 months, 15 months, 18 months, 21 months, or 24 months. In various embodiments, the patient is administered 240 mg total daily dose of sotorasib for at least 1 month. In various embodiments, the patient is administered 240 mg total daily dose of sotorasib for at least 3 months. In various embodiments, the patient is administered 240 mg total daily dose of sotorasib for at least 6 months.

The patient can respond to the sotorasib therapy as measured by at least a stable disease (SD), as determined by RECIST 1.1 protocol (Eisenhauer, et al., 2009). An at least stable disease is one that is a stable disease, has shown a partial response (PR) or has shown a complete response (CR) (i.e., “at least SD”=SD+PR+CR, often referred to as disease control). In various embodiments, the stable disease is neither sufficient shrinkage to qualify for partial response (PR) nor sufficient increase to qualify for progressive disease (PD). In various embodiments, the patient exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a patient's disease can be assessed by measuring tumor size, tumor lesions, or formation of new tumors or lesions, by assessing the patient using a computerized tomography (CT) scan, a positron emission tomography (PET) scan, a magnetic resonance imaging (MRI) scan, an X-ray, ultrasound, or some combination thereof.

Progression free survival can be assessed as described in the RECIST 1.1 protocol. In various embodiments, the patient exhibits a PFS of at least 3 months. In some embodiments, the patient exhibits a PFS of at least 6 months.

Additional means for assessing response are described in detail in the examples below and can generally be applied to the methods disclosed herein.

### KRAS G12C Cancers

Without wishing to be bound by any particular theory, the following is noted: sotorasib is a small molecule that specifically and irreversibly inhibits KRAS^{G12C} (Hong et al., 2020). Hong et al. report that “[p]reclinical studies showed that [sotorasib] inhibited nearly all detectable phosphorylation of extracellular signal-regulated kinase (ERK), a key down-stream effector of KRAS, leading to durable complete tumor regression in mice bearing KRAS p.G12C tumors.” (id., see also Canon et al., 2019, and Lanman et al., 2020). Thus, in various embodiments, sotorasib at a total daily dose of 240 mg is disclosed for use in treating cancer, wherein one or more cells express KRAS G12C mutant protein.

Sotorasib was evaluated in a Phase 1 dose escalation and expansion trial with 129 subjects having histologically confirmed, locally advanced or metastatic cancer with the KRAS G12C mutation identified by local molecular testing on tumor tissues, including 59 subjects with non-small cell lung cancer, 42 subjects with colorectal cancer, and 28 subjects with other tumor types (Hong et al., 2020, at page 1208-1209). Hong et al. report a disease control rate (95% CI) of 88.1% for non-small cell lung cancer, 73.8% for colorectal cancer and 75.0% for other tumor types (Hong et al., 2020, at page 1213, Table 3). The cancer types showing either stable disease (SD) or partial response (PR) as reported by Hong et al. were non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma (Hong et al., 2020, at page 1212 (Figure A), and Supplementary Appendix (page 59 (Figure S5) and page 63 (Figure S6)).

KRAS G12C mutations occur with the alteration frequencies shown in the table below (Cerami et al., 2012; Gao et al., 2013). For example, the table shows that 11.6% of subjects with non-small cell lung cancer have a cancer, wherein one or more cells express KRAS G12C mutant protein. Accordingly, sotorasib, which specifically and irreversibly bind to KRAS^{G12C} is useful for treatment of subjects having a cancer, including, but not limited to the cancers listed in Table 5 below.

TABLE 5

| | Alteration |
|---|---|
| Cancer Type | Frequency |
| Non-Small Cell Lung Cancer | 11.6 |
| Small Bowel Cancer | 4.2 |
| Appendiceal Cancer | 3.6 |
| Colorectal Cancer | 3.0 |
| Cancer of Unknown Primary | 2.9 |
| Endometrial Cancer | 1.3 |
| Mixed Cancer Types | 1.2 |
| Pancreatic Cancer | 1.0 |
| Hepatobiliary Cancer | 0.7 |
| Small Cell Lung Cancer | 0.7 |
| Cervical Cancer | 0.7 |
| Germ Cell Tumor | 0.6 |
| Ovarian Cancer | 0.5 |
| Gastrointestinal Neuroendocrine | 0.4 |
| Tumor | |
| Bladder Cancer | 0.4 |
| Myelodysplastic/Myeloproliferative | 0.3 |
| Neoplasms | |
| Head and Neck Cancer | 0.3 |
| Esophagogastric Cancer | 0.2 |
| Soft Tissue Sarcoma | 0.2 |
| Mesothelioma | 0.2 |
| Thyroid Cancer | 0.1 |
| Leukemia | 0.1 |
| Melanoma | 0.1 |

In various embodiments, the cancer is a solid tumor. In various embodiments, the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma. In some embodiments, the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma. In various embodiments, the cancer is non-small cell lung cancer, and in some specific embodiments, metastatic or locally advanced and unresectable non-small cell lung cancer. In various embodiments, the cancer is colorectal cancer. In some embodiments, the cancer is pancreatic cancer.

Methods of Detecting KRAS, STK11, KEAP1 EGFR, ALK, and/or ROS1 Mutation Status

The presence or absence of G12C, STK11, KEAP1, EGFR, ALK and/or ROS1 mutations in a cancer as described herein can be determined using methods known in the art. Determining whether a tumor or cancer comprises a mutation can be undertaken, for example, by assessing the nucleotide sequence encoding the protein, by assessing the amino acid sequence of the protein, or by assessing the characteristics of a putative mutant protein or any other suitable method known in the art. The nucleotide and amino acid sequences sequence of wild-type human KRAS (nucleotide sequence set forth in Genbank Accession No. BC010502; amino acid sequence set forth in Genbank Accession No. AGC09594), STK11 (Gene ID: 6794; available at https://www.ncbi.nlm.nih.gov/gene/6794; accessed January 2020), KEAP1 (Gene ID: 9817; available at www.ncbi.nlm.nih.gov/gene/9817; accessed January 2020), EGFR (Gene ID: 1956; available at www.ncbi.nlm.nih.gov/gene/1956; accessed March 2021), ALK (Gene ID: 238; available at https://www.ncbi.nlm.nih.gov/gene/238; accessed March 2021), and ROS1 (Gene ID: 6098; available at https://www.ncbi.nlm.nih.gov/gene/6098; accessed March 2021) are known in the art.

Methods for detecting a mutation include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct and/or next generation-based sequencing, primer extension reactions, electrophoresis, oligonucleotide ligation assays, hybridization assays, TagMan assays, SNP genotyping assays, high resolution melting assays and microarray analyses. In some embodiments, samples are evaluated for mutations, such as the KRAS G12C mutation, by real-time PCR. In real-time PCR, fluorescent probes specific for a certain mutation, such as the KRAS G12C mutation, are used. When a mutation is present, the probe binds and fluorescence is detected. In some embodiments, the mutation is identified using a direct sequencing method of specific regions in the gene. This technique identifies all possible mutations in the region sequenced. In some embodiments, gel electrophoresis, capillary electrophoresis, size exclusion chromatography, sequencing, and/or arrays can be used to detect the presence or absence of insertion mutations. In some embodiments, the methods include, but are not limited to, detection of a mutant using a binding agent (e.g., an antibody) specific for the mutant protein, protein electrophoresis and Western blotting, and direct peptide sequencing.

In some embodiments, multiplex PCR-based sequencing is used for mutation detection, and can include a number of amplicons that provides improved sensitivity of detection of one or more genetic biomarkers. For example, multiplex PCR-based sequencing can include about 60 amplicons (e.g., 50, 51, 52, 53, 54, 55, 56, 57, 58, 59, 60, 61, 62, 63, 64, 65, 66, 67, 68, 69, or 70 amplicons). In some embodiments, multiplex PCR-based sequencing can include 61 amplicons. Amplicons produced using multiplex PCR-based sequencing can include nucleic acids having a length from about 15 bp to about 1000 bp (e.g., from about 25 bp to about 1000 bp, from about 35 bp to about 1000 bp, from about 50 bp to about 1000 bp, from about 100 bp to about 1000 bp, from about 250 bp to about 1000 bp, from about 500 bp to about 1000 bp, from about 750 bp to about 1000 bp, from about 15 bp to about 750 bp, from about 15 bp to about 500 bp, from about 15 bp to about 300 bp, from about 15 bp to about 200 bp, from about 15 bp to about 100 bp, from about 15 bp to about 80 bp, from about 15 bp to about 75 bp, from about 15 bp to about 50 bp, from about 15 bp to about 40 bp, from about 15 bp to about 30 bp, from about 15 bp to about 20 bp, from about 20 bp to about 100 bp, from about 25 bp to about 50 bp, or from about 30 bp to about 40 bp). For example, amplicons produced using multiplex PCR-based sequencing can include nucleic acids having a length of about 33 bp.

In some embodiments, the presence of one or more mutations present in a sample obtained from a patient is detected using sequencing technology (e.g., a next-generation sequencing technology). A variety of sequencing technologies are known in the art. For example, methods for detection and characterization of circulating tumor DNA in cell-free DNA can be described elsewhere (see, e.g., Haber and Velculescu, 2014). Non-limiting examples of such techniques include SafeSeqs (see, e.g., Kinde et al., 2011), OnTarget (see, e.g., Forshew et al., 2012), and TamSeq (see, e.g., Thompson et al., 2012).

In some embodiments, the presence of one or more mutations present in a sample obtained from a patient is detected using droplet digital PCR (ddPCR), a method that is known to be highly sensitive for mutation detection. In some embodiments, the presence of one or more mutations present in a sample obtained from a patient is detected using other sequencing technologies, including but not limited to, chain-termination techniques, shotgun techniques, sequencing-by-synthesis methods, methods that utilize microfluidics, other capture technologies, or any of the other sequencing techniques known in the art that are useful for detection of small amounts of DNA in a sample (e.g., ctDNA in a cell-free DNA sample).

In some embodiments, the presence of one or more mutations present in a sample obtained from a patient is detected using array-based methods. For example, the step of detecting a genetic alteration (e.g., one or more genetic alterations) in cell-free DNA is performed using a DNA microarray. In some embodiments, a DNA microarray can detect one more of a plurality of cancer cell mutations. In some embodiments, cell-free DNA is amplified prior to detecting the genetic alteration. Non-limiting examples of array-based methods that can be used in any of the methods described herein, include: a complementary DNA (cDNA) microarray (see, e.g., Kumar et al. 2012; Laere et al. 2009; Mackay et al. 2003; Alizadeh et al. 1996), an oligonucleotide microarray (see, e.g., Kim et al. 2006; Lodes et al. 2009), a bacterial artificial chromosome (BAC) clone chip (see, e.g., Chung et al. 2004; Thomas et al. 2005), a single-nucleotide polymorphism (SNP) microarray (see, e.g., Mao et al. 2007; Jasmine et al. 2012), a microarray-based comparative genomic hybridization array (array-CGH) (see, e.g., Beers and Nederlof, 2006; Pinkel et al. 2005; Michels et al. 2007), a molecular inversion probe (MIP) assay (see, e.g., Wang et al. 2012; Lin et al. 2010). In some embodiments, the cDNA microarray is an Affymetrix microarray (see, e.g., Irizarry 2003; Dalma-Weiszhausz et al. 2006), a NimbleGen microarray (see, e.g., Wei et al. 2008; Albert et al. 2007), an Agilent microarray (see, e.g., Hughes et al. 2001), or a BeadArray array (see, e.g., Liu et al. 2017). In some embodiments, the oligonucleotide microarray is a DNA tiling array (see, e.g., Mockler and Ecker, 2005; Bertone et al. 2006). Other suitable array-based methods are known in the art.

Methods for determining whether a tumor or cancer comprises a mutation can use a variety of samples. In some embodiments, the sample is taken from a patient having a tumor or cancer. In some embodiments, the sample is a fresh tumor/cancer sample. In some embodiments, the sample is a frozen tumor/cancer sample. In some embodiments, the sample is a formalin-fixed paraffin-embedded (FFPE) sample. In some embodiments, the sample is a circulating cell-free DNA and/or circulating tumor cell (CTC) sample. In some embodiments, the sample is processed to a cell lysate. In some embodiments, the sample is processed to DNA or RNA. In a certain embodiment, the sample is acquired by resection, core needle biopsy (CNB), fine needle aspiration (FNA), collection of urine, or collection of hair follicles. In some embodiments, a liquid biopsy test using whole blood or cerebral spinal fluid may be used to assess mutation status.

In various embodiments, a test approved by a regulatory authority, such as the US Food and Drug Administration (FDA), is used to determine whether the patient has a mutation, e.g., a KRASG¹²° mutated cancer, or whether the tumor or tissue sample obtained from such patient contains cells with a mutation. In some embodiments, the test for a KRAS mutation used is Therascreen® KRAS RGQ PCR Kit (Qiagen). The Therascreen® KRAS RGQ PCR Kit is a real-time qualitative PCR assay for the detection of 7 somatic mutations in codons 12 and 13 of the human KRAS oncogene (G12A, G12D, G12R, G12C, G125, G12V, and G13D) using the Rotor-Gene Q MDx 5plex HRM instrument. The kit is intended for use with DNA extracted from FFPE samples of NSCLC samples acquired by resection, CNB, or FNA. Mutation testing for STK11, KEAP1, EGFR, ALK and/or ROS1 can be conducted with commercially available tests, such as the Resolution Bioscience Resolution ctDx Lung™ assay that includes 24 genes (including those actionable in NSCLC). Tissue samples may be tested using Tempus xT 648 panel.

In some embodiments, the cancer has been identified as having a KRAS G12C mutation. In some embodiments, the cancer has been identified as having a mutation of STK11, e.g., a loss-of-function mutation. In some embodiments, the cancer has been identified as having a mutation of KEAP1, e.g., a loss-of-function mutation. In some embodiments, the cancer has been identified as having wild-type STK11. In some embodiments, the cancer has been identified as having wild-type KEAP1.

In various embodiments, the cancer has been identified as having a loss-of-function mutation of STK11 and wild-type KEAP1. In some embodiments, the cancer has been identified as having a loss-of-function mutation of STK11 and a loss-of-function mutation of KEAP1. In some embodiments, the cancer has been identified as having wild-type of STK11 and wild-type KEAP1. In some embodiments, the cancer has been identified as having wild type of STK11 and a loss-of-function mutation of KEAP1.

The term “loss-of-function mutation” as used herein refers to a mutation (e.g., a substitution, deletion, truncation, or frameshift mutation) that results in expression of a mutant protein that no longer exhibits wild-type activity (e.g., reduced or eliminated wild-type biological activity or enzymatic activity), results in expression of only a fragment of the protein that no longer exhibits wild-type activity, or results in no expression of the wild-type protein. For example, a loss-of-function mutation affecting the STK11 gene in a cell may result in the loss of expression of the STK11 protein, expression of only a fragment of the STK11 protein, or expression of the STK11 protein that exhibits diminished or no enzymatic activity (e.g., no serine/threonine kinase enzymatic activity) in the cancerous cell. Similarly, a loss-of-function mutation affecting the KEAP1 gene in a cell may result in the loss of expression of the KEAP1 protein, expression of only a fragment of the KEAP1 protein, or expression of a KEAP1 protein that exhibits diminished or no activity (e.g., inability to interact with or activate Nuclear factor erythroid 2-related factor 2 (NRF2)) in the cell.

### Methods of Detecting PDL1 Protein Expression

PDL1 expression can be determined by methods known in the art. For example, PDL1 expression can be detected using PDL1 IHC 22C3 pharmDx, an FDA-approved in vitro diagnostic immunohistochemistry (IHC) test developed by Dako and Bristol-Meyers Squibb as a companion test for treatment with pembrolizumab. This is qualitative assay using Monoclonal Mouse Anti-PD-L1, Clone 22C3 PDL1 and EnVision FLEX visualization system on Autostainer Lin 48 to detect PDL1 in FFPE samples, such as human non-small cell lung cancer tissue. Expression levels can be measured using the tumor proportion score (TPS), which measures the percentage of viable tumor cells showing partial or complete membrane staining at any intensity. Staining can show PDL1 expression from 0% to 100%.

PDL1 expression can also be detected using PDL1 IHC 28-8 pharmDx, the FDA-approved in vitro diagnostic immunohistochemistry (IHC) test developed by Dako and Merck as a companion test for treatment with nivolumab. This qualitative assay uses the Monoclonal rabbit anti-PDL1, Clone 28-8 and EnVision FLEX visualization system on Autostainer Lin 48 to detect PDL1 in formalin-fixed, paraffin-embedded (FFPE) human cancer tissue.

Other commercially available tests for PDL1 detection include the Ventana SP263 assay (developed by Ventana in collaboration with AstraZeneca) that utilizes monoclonal rabbit anti-PD-LI, Clone SP263 and the Ventana SP142 Assay (developed by Ventana in collaboration with Genentech/Roche) that uses rabbit monoclonal anti-PDL1 clone SP142.

In some embodiments, a test approved by a regulatory authority, such as the US Food and Drug Administration (FDA), is used to determine the PDL1 TPS of a cancer as disclosed herein. In various embodiment, the PDL1 TPS is determined using a immunohistochemistry (IHC) test. In some embodiments, the IHC test is the PDL1 IHC 22C3 pharmDx test. In various embodiments, the IHC test conducted with samples acquired by, for example, resection, CNB, or FNA.

In various embodiment, the patient has a PDL1 TPS of less than 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PDL1 TPS of less than 50%, or less than 1%. In various embodiments, the patient has a PDL1 TPS of more than or equal to 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PDL1 TPS of less than or equal to 100%, 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PDL1 TPS of less than or equal to 50%, or less than or equal to 1%. In various embodiments, the patient has a PDL1 TPS of more than 95%, 90%, 85%, 80%, 75%, 70%, 65%, 60%, 50%, 55%, 50%, 45%, 40%, 35%, 30%, 25%, 20%, 15%, 10%, 9%, 8%, 7%, 6%, 5%, 4%, 3%, 2%, or 1%. In various embodiments, the patient has a PDL1 TPS score a range bound by any of the values cited in the foregoing embodiments. For example, the patient has a PDL1 TPS score in the range of less than 50% and more than or equal to 1%, less than or equal to 50% and more than 1%, less than or equal to 50% and more than or equal to 1%, or less than 50% and more than 1%.

In various embodiments, the patient has a PDL1 TPS score in the range of less than 50% and more than or equal to 1%. In some embodiments, the patient has a PDL1 TPS score in the range of more than or equal to 0% and less than 1%. In some embodiments, the patient has a PDL1 TPS score in the range of more than 50% and less than or equal to 100%. In some embodiments, the patient has a PDL1 TPS score of less than 1%. In some embodiments, the patient as a PDL1 TPS score of 1-49%. In some embodiments, the patient has a PDL1 TPS score of 50% or greater (i.e., 50%-100%).

### Embodiments

1. A method of treating cancer in a patient comprising administering a total daily dose of 240 mg sotorasib to the patient, wherein the cancer is a KRAS p G12C mutated cancer.

2. A method of treating cancer in a patient comprising administering an initial total daily dose of 960 mg sotorasib to the patient, and administering a reduced total daily dose of sotorasib of 480 mg when the patient experiences an adverse event to the initial total daily dose, wherein the cancer is a KRAS p G12C mutated cancer.

3. The method of embodiment 2, further comprising administering a second reduced total daily dose of sotorasib of 240 mg when the patient experiences an adverse event to the reduced total daily dose.

4. The method of embodiments 2 or 3, wherein the adverse event is an elevation of one or more liver enzymes in the patient, wherein the liver enzyme is alanine aminotransferase (ALT) or aspartate aminotransferase (AST).

5. The method of embodiment 4, wherein the elevated level of ALT and/or AST is >3×ULN.

6. The method of any one of embodiments 2-5, further comprising withholding sotorasib treatment from the patient until ALT and/or AST levels in the patient improve to Grade 1 or to baseline before administering the reduced total daily dose of sotorasib or the second reduced total daily dose of sotorasib.

7. The method of any one of embodiments 2-6, comprising discontinuing sotorasib treatment when levels of AST or ALT>3×ULN with total bilirubin>2×ULN in the absence of alternative causes.

8. The method of any one of embodiments 2-7, wherein the adverse event is diarrhea.

9. The method of embodiment 8, further comprising withholding sotorasib treatment from the patient until diarrhea in the patient improves to Grade 1 or to baseline before administering the reduced total daily dose of sotorasib or the second reduced total daily dose of sotorasib.

10. The method of any one of embodiments 2-9, wherein the adverse event is nausea/vomiting.

11. The method of embodiment 10, further comprising withholding sotorasib treatment from the patient until nausea/vomiting in the patient improves to Grade 1 or to baseline before administering the reduced total daily dose of sotorasib or the second reduced total daily dose of sotorasib.

12. The method of any one of embodiments 1-11, wherein the sotorasib is administered once per day.

13. The method of any one of embodiments 1-12, wherein the sotorasib is administered orally.

14. The method of any one of embodiments 1-13, wherein the cancer is a solid tumor.

15. The method of any one of embodiments 1-14, wherein the cancer is non-small cell lung cancer.

16. The method of embodiment 15, wherein the cancer is metastatic non-small cell lung cancer.

17. The method of embodiment 16, wherein the cancer is locally advanced and unresectable.

18. The method of any one of embodiments 1-13, wherein the cancer is colorectal cancer.

19. The method of any one of embodiments 1-13, wherein the cancer is pancreatic cancer.

20. The method of any one of embodiments 1-13, wherein the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma.

21. The method of any one of embodiments 1-20, wherein the patient, prior to start of sotorasib therapy, had undergone at least one other systemic cancer therapy.

22. The method of embodiment 21, wherein the patient had undergone at least two other systemic cancer therapies.

23. The method of embodiment 21 or 22, wherein at least one systemic cancer therapy is selected from anti-PD1 immunotherapy, anti-PDL1 immunotherapy, and platinum-based chemotherapy.

24. The method of embodiment 23, wherein the patient has previously undergone (i) an anti-PD1 therapy or anti-PDL1 therapy, unless contraindicated, or (ii) a platinum-based chemotherapy, and (iii) a EGFR, ALK or ROS1 targeted therapy if the cancer also exhibited a mutation in EGFR, ALK, or ROS1.

25. The method of embodiment 23, wherein the patient has previously undergone (i) an anti-PD1 therapy or anti-PDL1 therapy, unless contraindicated, and (ii) a platinum-based chemotherapy, and (iii) a EGFR, ALK or ROS1 targeted therapy if the cancer also exhibited a mutation in EGFR, ALK, or ROS1.

26. The method of any one of embodiments 1-25, wherein the patient does not have brain metastases within four weeks of the start of sotorasib therapy.

27. The method of any one of embodiments 1-26, wherein the patient exhibits an Eastern Cooperative Oncology Group (ECOG) performance status of 0, 1, or 2.

28. The method of any one of embodiments 1-27, wherein the patient is administered sotorasib for at least one month.

29. The method of any one of embodiments 1-27, wherein the patient is administered sotorasib for at least three months.

30. The method of any one of embodiments 1-27, wherein the patient is administered sotorasib for at least six months.

31. The method of any one of embodiments 28-30, wherein the patient exhibits at least a stable disease (SD) after 1, 3, or 6 months of sotorasib therapy, as measured by RECIST 1.1 protocol.

32. The method of embodiment 31, wherein the stable disease is neither sufficient shrinkage to qualify for partial response (PR) nor sufficient increase to qualify for progressive disease (PD).

33. The method of any one of embodiments 28-31, wherein the patient exhibits at least a partial response (PR) after 1, 3, or 6 months of sotorasib therapy, as measured by RECIST 1.1 protocol.

34. The method of embodiment 33, wherein the partial response is at least a 30% decrease in the sum of diameters of target lesions.

35. The method of any one of embodiments 1-34, wherein the patient exhibits a progression free survival (PFS) of at least 3 months.

36. The method of embodiment 35, wherein the patient exhibits a PFS of at least 6 months.

37. The method of any one of embodiments 1-36, wherein the cancer exhibits a PDL1 tumor proportion score (TPS) of 1-49%.

38. The method of any one of embodiments 1-36, wherein the cancer exhibits a PDL1 tumor proportion score (TPS) of less than 1%.

39. The method of any one of embodiments 1-36, wherein the cancer exhibits a PDL1 tumor proportion score (TPS) of 50-100%.

40. The method of any one of embodiments 1-39, wherein the cancer further comprises a STK11 mutation.

41. The method of any one of embodiments 1-40, wherein the cancer further comprises a KEAP1 mutation.

42. The method of any one of embodiments 1-39 and 41, wherein the cancer further comprises a STK11 wild type.

43. The method of any one of embodiments 1-40 and 42, wherein the cancer further comprises a KEAP1 wild type.

44. The method of any one of embodiments 1-43, wherein the patient exhibits hepatotoxicity and the method further comprises administering a steroid to the patient.

45. The method of embodiment 44, wherein the steroid is prednisone at a dose of 0.25 to 1.0 mg/kg/day.

46. The method of any one of embodiments 1-45, wherein the patient is in further need of treatment with an acid-reducing agent.

47. The method of embodiment 46, wherein the acid-reducing agent is a proton pump inhibitor (PPI), a H2 receptor antagonist (H2RA), or a locally acting antacid.

48. The method of embodiment 46 or embodiment 47, provided the patient is in further need of treatment with an acid reducing agent, sotorasib is administered about 4 hours before or about 10 hours after the locally acting antacid.

49. The method of any one of embodiments 46-48, wherein the locally acting antacid is sodium bicarbonate, calcium carbonate, aluminum hydroxide, or magnesium hydroxide.

50. The method of any one of embodiments 1-45, wherein the patient is in further need of treatment with a proton pump inhibitor (PPI) or H2 receptor antagonist (H2RA).

51. The method of embodiment 47, wherein the patient is not administered a PPI or a H2RA in combination with sotorasib.

52. The method of any one of embodiments 47, or 50-51, wherein the PPI is omeprazole, pantoprazole, esomeprazole, lansoprazole, rabeprazole, or dexlansoprazole.

53. The method of any one of embodiments 47, or 50-51, wherein the H2RA is famotidine, ranitidine, cimetidine, nizatidine, roxatidine or lafutidine.

54. The method of any one of embodiments 1-53, wherein the patient is in further need of treatment with a CYP3A4 inducer.

55. The method of embodiment 54, wherein the patient is not administered a CYP3A4 inducer in combination with sotorasib.

56. The method of embodiment 54 or 55, wherein the CYP3A4 inducer is barbiturates, brigatinib, carbamazepine, clobazam, dabrafenib, efavirenz, elagolix, enzalutamide, eslicarbazepine, glucocorticoids, letermovir, lorlatinib, modafinil, nevirapine, oritavancin, oxcarbazepine, perampanel, phenobarbital, phenytoin, pioglitazone, rifabutin, rifampin, telotristat, and troglitazone.

57. The method of embodiment 54, wherein the patient is not administered a strong CYP3A4 inducer in combination with sotorasib.

58. The method of embodiment 57, wherein the strong CYP3A4 inducer is phenytoin or rifampin.

59. The method of any one of embodiments 1-58, wherein the patient is in further need of treatment with a CYP3A4 substrate.

60. The method of embodiment 59, wherein the patient is not administered a CYP3A4 substrate in combination with sotorasib.

61. The method of embodiment 59 or 60, wherein the CYP3A4 substrate is abemaciclib, abiraterone, acalabrutinib, alectinib, alfentanil, alprazolam, amitriptyline, amlodipine, apixaban, aprepitant, aripiprazole, astemizole, atorvastatin, avanafil, axitinib, boceprevir, bosutinib, brexpiprazole, brigatinib, buspirone, cafergot, caffeine, carbamazepine, cariprazine, ceritinib, cerivastatin, chlorpheniramine, cilostazol, cisapride, citalopram, clarithromycin, clobazam, clopidogrel, cobimetinib, cocaine, codeine, colchicine, copanlisib, crizotinib, cyclosporine, dabrafenib, daclatasvir, dapsone, deflazacort, dexamethasone, dextromethorphan, diazepam, diltiazem, docetaxel, dolutegravir, domperidone, doxepin, elagolix, elbasvir/grazoprevir, eliglustat, enzalutamide, eplerenone, erythromycin, escitalopram, esomeprazole, estradiol, felodipine, fentanyl, finasteride, flibanserin, gleevec, haloperidol, hydrocortisone, ibrutinib, idelalisib, indacaterol, indinavir, irinotecan, isavuconazonium, ivabradine, ivacaftor, lansoprazole, lenvatinib, lercanidipine, lidocaine, linagliptin, lovastatin, macitentan, methadone, midazolam, naldemedine, naloxegol, nateglinide, nelfinavir, neratinib, netupitant/palonosetron, nevirapine, nifedipine, nisoldipine, nitrendipine, olaparib, omeprazole, ondansetron, osimertinib, ospemifene, palbociclib, panobinostat, pantoprazole, perampanel, pimavanserin, pimozide, pomalidomide, ponatinib, progesterone, propranolol, quetiapine, quinidine, quinine, regorafenib, ribociclib, rilpivirine, risperidone, ritonavir, rivaroxaban, roflumilast, rolapitant, romidepsin, ruxolitinib, salmeterol, saquinavir, selexipag, sildenafil, simeprevir, simvastatin, sirolimus, sonidegib, sorafenib, sunitinib, suvorexant, tacrolimus(fk506), tamoxifen, tasimelteon, taxol, telaprevir, telithromycin, terfenadine, testosterone, ticagrelor, tofacitinib, tolvaptan, torisel, tramadol, trazodone, valbenazine, vandetanib, velpatasvir, vemurafenib, venetoclax, venlafaxine, verapamil, vilazodone, vincristine, vorapaxar, voriconazole, zaleplon, and ziprasidone.

62. The method of any one of embodiments 1-61, wherein the patient is in further need of treatment with a P-glycoprotein (P-gp) substrate.

63. The method of embodiment 62, wherein the patient is not administered a P-gp substrate in combination sotorasib.

64. The method of embodiment 57 or embodiment 58, wherein the P-gp substrate is etexilate, digoxin, and fexofenadine.

### EXAMPLES

### Example 1—Pharmacokinetic Analysis of 960 mg, 360 mg, 180 mg, and 240 mg Sotorasib

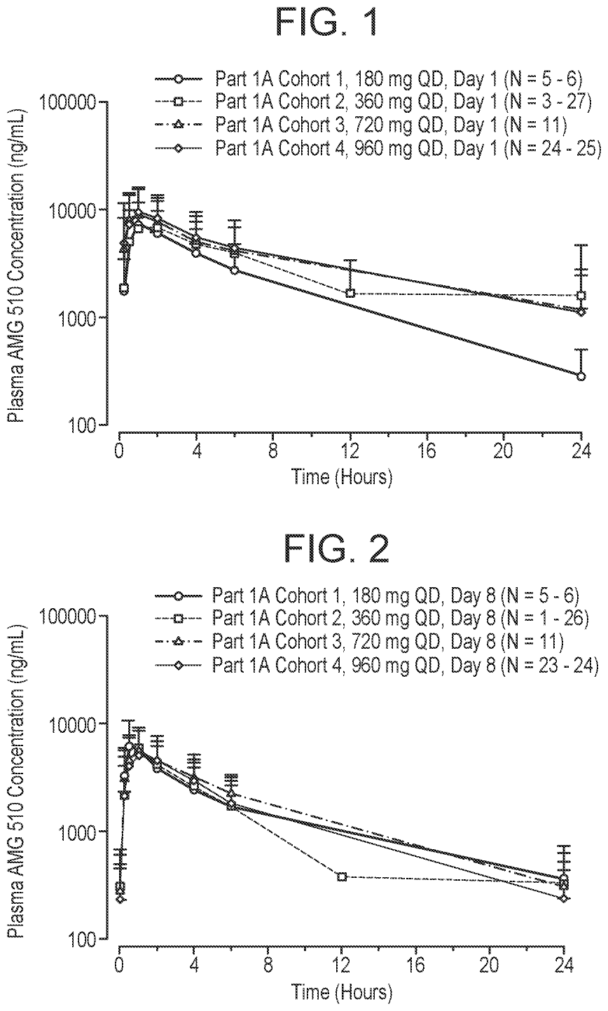

Preliminary pharmacokinetic (PK) data were available for subjects with advanced solid tumors with the specific KRAS p.G12C mutation, with doses ranging from 180 to 960 mg PO QD. Dose-related increases in exposure on day 1 from 180 to 960 mg PO QD were observed. Increases in exposure were less than dose-proportional on day 1. There was no accumulation with multiple PO QD dosing for 8 days. The change in exposure from 180 to 960 mg PO QD was less than dose-proportional on day 8. Rapid absorption was observed with tmax between 1 to 2 hours after PO administration. FIG. 1 shows the mean plasma concentration time profile after oral administration of 180, 360, 720, or 960 mg sotorasib on Day 1. FIG. 2 shows the concentrations after once daily dosing for 8 days (Day 8). The table below provides the pharmacokinetic parameters, where AUC₀₋₂₄ₕ is the area under the concentration-time curve from time 0 to 24 hr postdose; Cₘₐₓ is the maximum observed drug concentration during a dosing interval; t_{1/2,z} is the terminal elimination half-life; tₘₐₓ is the time to reach Cₘₐₓ. Data reported are presented as geometric mean (arithmetic CV %) except tₘₐₓ and t_{1/2}, which are reported as a median (range) and arithmetic mean (SD), respectively. Values are reported to three significant figures, except CV % and tₘₐₓ, which are reported to 0 decimal places and 2 significant figures, respectively.

TABLE 6

| | Pharmacokinetic Parameter | | | | | | | |
|---|---|---|---|---|---|---|---|---|
| | | tₘₐₓ | | Cₘₐₓ | AUC_{0-24 h} | | t_{1/2, z} | |
| Dose | N | (hr) | | (μg/mL) | (hr · μg/mL) | | (hr) | |
| 180 mg | | | | | | | | |
| Day 1 | 6 | 1.0 | (0.50-2.0) | 6.88 (51%) | 44.0 | (56%) | 5.95 | (1.08)^{a} |
| Day 8 | 6 | 0.75 | (0.50-1.0) | 6.44 (67%) | 33.5 | (85%) | 5.96 | (2.76)^{b} |
| 360 mg | | | | | | | | |
| Day 1 | 25 | 1.0 | (0.50-24) | 5.86 (68%) | 56.8 | (84%) | 6.56 | (1.81)^{c} |
| Day 8 | 25 | 1.0 | (0.25-4.0) | 5.97 (46%) | 37.4 | (50%) | 5.71 | (1.59)^{d} |
| 720 mg | | | | | | | | |
| Day 1 | 11 | 1.0 | (0.50-4.0) | 7.57 (59%) | 64.0 | (68%) | 7.06 | (1.59)^{e} |
| Day 8 | 11 | 1.0 | (0.50-4.0) | 5.45 (50%) | 43.9 | (49%) | 5.06 | (1.24)^{f} |
| 960 mg | | | | | | | | |
| Day 1 | 25 | 2.0 | (0.25-6.0) | 8.33 (59%) | 68.0 | (77%) | 6.00 | (2.20)^{g} |
| Day 8 | 25 | 1.0 | (0.50-24) | 4.91 (69%) | 32.7 | (70%)^{h} | 5.19 | (1.12)ⁱ |

^{a}N = 5;
^{b}N = 6;
^{c}N = 17;
^{d}N = 19;
^{e}N = 8;
^{f}N = 9;
^{g}N = 18;
^{h}N = 24;
ⁱN = 16;

### Example 2—Efficacy of 180 mg, 360 mg, and 720 mg QD in Non-Small Cell Lung Cancer

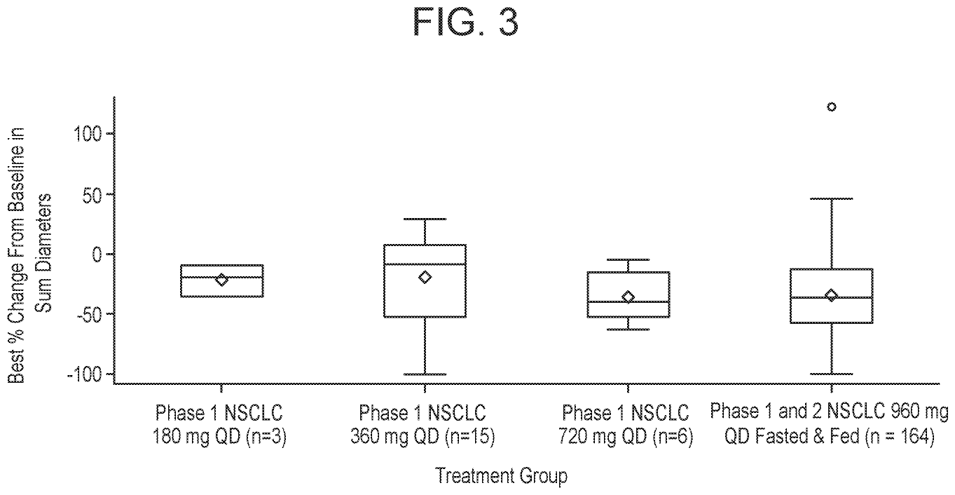

Patients diagnosed with non-small cell lung cancer or other solid tumors and determined to have a KRAS^{G12C} mutation were administered 180 mg, 360 mg, 720 mg, and 960 mg QD sotorasib orally. Responses were seen across all dose levels studied (Hong et al., 2020). A boxplot of best tumor shrinkage for NSCLC patients given 180 mg QD, 360 mg QD, 720 mg QD, and 960 mg QD is shown in FIG. 3.

Of 24 NSCLC patients evaluable and treated at doses 180 mg, 360 mg, or 720 mg, responses were seen in 8 patients (an ORR of 33.3%). Of the 34 NSCLC patients treated with 960 mg, responses were observed in 12 subjects (an ORR of 35.5%). In a phase 2 study of sotorasib 960 mg QD, the ORR in NSCLC (N=124) was 37.1%.

The objective response of NSCLC patients is shown in Table 7 below:

TABLE 7

| | 180 mg QD | | 360 mg QD | | 720 mg QD | | 960 mg QD | |
|---|---|---|---|---|---|---|---|---|
| | (N = 3) | | (N = 16) | | (N = 6) | | (N = 169) | |
| Best overall response - n (%) | | | | | | | | |
| Complete response (CR) | | | | | | | | |
| Confirmed | 0 | (0.0) | 0 | (0.0) | 0 | (0.0) | 3 | (1.8) |
| Confirmed and unconfirmed | 0 | (0.0) | 0 | (0.0) | 0 | (0.0) | 4 | (2.4) |
| awaiting confirmatory scan | | | | | | | | |
| Partial response (PR) | | | | | | | | |
| Confirmed | 1 | (33.3) | 4 | (25.0) | 3 | (50.0) | 61 | (36.1) |
| Confirmed and unconfirmed | 1 | (33.3) | 4 | (25.0) | 3 | (50.0) | 63 | (37.3) |
| awaiting confirmatory scan | | | | | | | | |
| Stable disease (SD) | 2 | (66.7) | 10 | (62.5) | 3 | (50.0) | 77 | (45.6) |
| Progressive disease (PD) | 0 | (0.0) | 1 | (6.3) | 0 | (0.0) | 22 | (13.0) |
| Not evaluable (NE) | 0 | (0.0) | 0 | (0.0) | 0 | (0.0) | 2 | (1.2) |
| Not done | 0 | (0.0) | 1 | (6.3) | 0 | (0.0) | 4 | (2.4) |
| Objective response rate (ORR) | | | | | | | | |
| Confirmed - N1 (%) | 1 | (33.3) | 4 | (25.0) | 3 | (50.0) | 64 | (37.9) |
| 95% CI^{a} | (0.84, 90.57) | | (7.27, 52.38) | | (11.81, 88.19) | | (30.53, 45.64) | |
| Confirmed and unconfirmed awaiting | 1 | (33.3) | 4 | (25.0) | 3 | (50.0) | 67 | (39.6) |
| confirmatory scan - n (%) | | | | | | | | |
| 95% CI^{a} | (0.84, 90.57) | | (7.27, 52.38) | | (11.81, 88.19) | | (32.22, 47.44) | |
| Disease control rate (DCR) - n (%) | 3 | (100.0) | 14 | (87.5) | 6 | (100.0) | 141 | (83.4) |
| 95% CI^{a} | (29.24, 100.00) | | (61.65, 98.45) | | (54.07, 100.00) | | (76.95, 88.70) | |
| Duration of objective response | | | | | | | | |
| (DOR)^{b} | | | | | | | | |
| Observed duration ≥3 months - n | 1 | (100.0) | 4 | (100.0) | 2 | (66.7) | 47 | (73.4) |
| (%) | | | | | | | | |
| Observed duration ≥6 months - n | 1 | (100.0) | 2 | (50.0) | 2 | (66.7) | 28 | (43.8) |
| (%) | | | | | | | | |
| Observed duration ≥9 months - n | 1 | (100.0) | 1 | (25.0) | 1 | (33.3) | 4 | (6.3) |
| (%) | | | | | | | | |
| Observed duration ≥12 months - n | 0 | (0.0) | 1 | (25.0) | 0 | (0.0) | 1 | (1.6) |
| (%) | | | | | | | | |

Months are derived as days x (12/365.25).
^{a}Exact 95% confidence interval was calculated using the Clopper Pearson method.
^{b}Time to response and duration of response are calculated among confirmed responders N1.

### Example 3—Dose Comparison Study of 960 mg QD and 240 mg QD

Sotorasib at 960 mg QD was shown to be safe and effective under study conditions under Study 20170543 (CodeBreak100). However, sotorasib demonstrates a non-linear pharmacokinetic profile in human, with responses noted at all dose levels ranging from 180 mg to 960 mg. Based upon the observed pharmacokinetic profile discussed in Example 1, the 240 mg QD dose is expected to approximate the exposure at a lower dose of 180 mg or 360 mg QD. Drug exposure at the 240 mg QD dose is expected to similar to the 960 mg QD dose and the 240 mg QD dose is expected to be above the concentration associated with 90% inhibition in vitro in 2 hour cellular pERK assay (see, e.g., Hong et al. 2020, Supplementary Appendix, Figure S3).

A multicenter, randomized, open-label study is set up to evaluate the safety and efficacy of sotorasib as monotherapy in subjects with previously treated locally advanced and unresectable or metastatic KRAS G12C mutant advanced NSCLC. Approximately 200 subjects are enrolled and randomized 1:1 to receive sotorasib at 960 mg QD or 240 mg QD. Tumor response is evaluated employing RECIST 1.1 based on contrast enhanced CT/MRI with assessments conducted by an independent radiological central laboratory. Subjects continue treatment until disease progression, intolerance of treatment leading to treatment discontinuation, initiation of another anticancer therapy or withdrawal of consent. Subjects' scans undergo independent central confirmation of progression (COP) at the time of first progressive disease (PD). After centrally confirmed progression, subjects in both arms have an option to continue sotorasib therapy at their current dose if tolerable and no reasonable alternative treatment options are available in the opinion of the investigator. Subjects that undergo treatment beyond progression continue to receive scans after confirmation of first PD.

### Subject Inclusion Criteria Include the Following:

Subject has provided informed consent prior to initiation of any study specific activities/procedures

Men or women at least 18 years old.

Pathologically documented, locally-advanced or metastatic malignancy with KRAS G12C mutation identified through molecular testing.

For NSCLC: subjects must have progressed after receiving anti-PD1 or anti PDL1 immunotherapy (unless contraindicated) AND/OR platinum based combination chemotherapy AND targeted therapy if actionable oncogenic driver mutations were identified (i.e., EGFR, ALK, and ROS1).

For all NSCLC subjects, the following guidance should be used: (1) Adjuvant therapy counts as a line of therapy if the subject progressed on or within 6 months of adjuvant therapy administration. (2) In locally advanced and unresectable NSCLC, disease progression on or within six months of end of prior curatively intended multimodal therapy counts as a line of therapy. If chemoradiation is followed by planned systemic therapy without documented progression between chemoradiation and systemic therapy, the entire treatment course counts as 1 line of therapy. (3) Maintenance therapy following platinum doublet-based chemotherapy is not considered as a separate line of therapy.

For CRC: subjects must have progressed after receiving fluoropyrimidine AND oxaliplatin AND irinotecan. For those CRC subjects with tumors that are MSI-H, at least 1 of the prior systemic regimens must have included an anti-PD1 therapy if they were clinically able to receive inhibitors and 1 of these agents is approved for that indication in the region or country.

For advanced solid tumor types other than NSCLC or CRC, subjects must have received at least 1 prior systemic therapy or be intolerant or ineligible for available therapies known to provide clinical benefit. Subjects with advanced solid tumor types other than NSCLC or CRC may be enrolled and treated in phase 1 or phase 2 without central confirmation of the KRAS p.G12C mutation.

Subjects willing to provide archived tumor tissue samples (formalin fixed, paraffin embedded [FFPE] sample collected within 5 years) or willing to undergo pretreatment tumor biopsy. subjects with tumor types other than NSCLC or CRC with prior molecularly confirmed KRAS p.G12C mutation who do not have archived tissue available can be allowed to enroll without undergoing tumor biopsy upon agreement with investigator and the Medical Monitor if a tumor biopsy is not feasible.

Subjects who have lesions that can be feasibly biopsied will be asked to undergo an optional biopsy at the time of tumor progression.

Measurable disease per RECIST 1.1 criteria.

Eastern Cooperative Oncology Group (ECOG) Performance Status of 2.

Adequate renal laboratory assessments, as follows: Estimated glomerular filtration rate based on MDRD (Modification of Diet in Renal Disease) calculation 45 ml/min/1.73 m².

### Exclusion Criteria Include the Following:

Active brain metastases from non-brain tumors. Subjects who have had brain metastases resected or have received radiation therapy ending at least 4 weeks prior to study day 1 are eligible if they meet all of the following criteria: a) residual neurological symptoms grade 2; b) on stable doses of dexamethasone, if applicable; and c) follow-up MRI performed within 30 days shows no new lesions appearing.

History or presence of hematological malignancies unless curatively treated with no evidence of disease [symbol] 2 years.

Myocardial infarction within 6 months of study day 1, symptomatic congestive heart failure (New York Heart Association>class II), unstable angina, or cardiac arrhythmia requiring medication.

Gastrointestinal (GI) tract disease causing the inability to take oral medication, malabsorption syndrome, requirement for intravenous alimentation, uncontrolled inflammatory GI disease (e.g., Crohn's disease, ulcerative colitis).

Active infection requiring IV antibiotics within 1 weeks of study enrollment (day 1).

Exclusion of hepatitis infection based on the following results and/or criteria: Positive Hepatitis B Surface Antigen (HepBsAg) (indicative of chronic Hepatitis B or recent acute hepatitis B); Negative HepBsAg with a positive for hepatitis B core antibody (Hepatitis B core antibody testing is not required for screening, however if this is done and is positive, then hepatitis B surface antibody [Anti-HBs] testing is necessary. Undetectable anti-HBs in this setting would suggest unclear and possible infection, and needs exclusion); Positive Hepatitis C virus antibody: Hepatitis C virus RNA by PCR is necessary. Detectable Hepatitis C virus RNA suggests chronic hepatitis C.

Known positive test for HIV.

Unresolved toxicities from prior anti-tumor therapy, defined as not having resolved to Common Terminology Criteria for Adverse Events (CTCAE) version 5.0 grade 0 or 1, or to levels dictated in the eligibility criteria with the exception of alopecia (grade 2 or 3 toxicities from prior anti-tumor therapy that are considered irreversible [defined as having been present and stable for >6 months], such as ifosfamide-related proteinuria, may be allowed if they are not otherwise described in the exclusion criteria AND there is agreement to allow by both the investigator and sponsor.

Anti-tumor therapy (chemotherapy, antibody therapy, molecular targeted therapy, retinoid therapy, hormonal therapy [except for subjects with breast cancer], or investigational agent) within 28 days of study day 1; concurrent use of hormone deprivation therapy for hormone-refractory prostate cancer or breast cancer is permitted.

Therapeutic or palliative radiation therapy within 2 weeks of study day 1. Subjects must have recovered from all radiotherapy related toxicity.

Currently enrolled in another investigational device or drug study, or less than 28 days since ending another investigational device or drug study(s), or receiving other investigational agent(s). Exception: subjects enrolled in the long-term follow-up portion of another investigational device or drug study but not taking the respective investigational drug or device.

Other investigational procedures are excluded.

Major surgery within 28 days of study day 1.

Monotherapy with AMG 510: Men and women of childbearing potential (WOCBP) who are unwilling to practice acceptable methods of birth control during treatment and for at least 7 days (women) or 7 days (men) after receiving the last dose of AMG 510. Acceptable methods of highly effective birth control for women include sexual abstinence (refraining from heterosexual intercourse); vasectomy (women with a single male sexual partner) with testing showing there is no sperm in the semen; bilateral tubal ligation or occlusion; or intrauterine device. Acceptable methods of birth control for men include sexual abstinence (refraining from heterosexual intercourse); vasectomy with testing showing there is no sperm in the semen; bilateral tubal ligation or occlusion in the partner; or a condom (the female partner should also consider a form of birth control). Note: A woman is considered of childbearing potential (WOCBP), i.e., fertile, following menarche and until becoming postmenopausal unless permanently sterile. Permanent sterilization methods include hysterectomy, bilateral salpingectomy, and bilateral oophorectomy. A postmenopausal state is defined as no menses for 12 months without an alternative medical cause. A high follicle stimulating hormone level in the postmenopausal range may be used to confirm a postmenopausal state in women not using hormonal contraception or hormonal replacement therapy. However, in the absence of 12 months of amenorrhea, a single follicle stimulating hormone measurement is insufficient.

Women who are lactating/breast feeding or who plan to breastfeed while on study through 7 days after receiving the last dose of study drug.

Women with a positive pregnancy test.

Women planning to become pregnant while on study through 7 days after receiving the last dose of study drug.

Subject has known sensitivity to any of the products to be administered during dosing.

Subject will not be available for protocol-required study visits or procedures, to the best of the subject and investigator's knowledge.

Subject has any kind of disorder that, in the opinion of the investigator, may compromise the ability of the subject to give written informed consent and/or to comply with all required study procedures.

History or evidence of any other clinically significant disorder, condition or disease (with the exception of those outlined above) that, in the opinion of the investigator or company physician would pose a risk to subject safety or interfere with the study evaluation, procedures or completion.

Use of known P-gp sensitive substrates (with a therapeutic window), within 14 days or 5 half-lives of the drug or its major active metabolite, whichever is longer, prior to study day 1 that was not reviewed and approved by the principal investigator.

Use of proton-pump inhibitors (PPIs) of H2 receptor antagonists within 14 days or 5 half-lives of the drug or its major active metabolite, whichever is longer, prior to study day 1 that was not reviewed and approved by the principal investigator.

Use of known cytochrome P450 (CYP) 3A4 sensitive substrates (with a narrow therapeutic window), within 14 days or 5 half-lives of the drug or its major active metabolite, whichever is longer, prior to study day 1 that was not reviewed and approved by the principal investigator and the company medical monitor.

Use of strong inducers of CYP3A4 (including herbal supplements such as St. John's wort) within 14 days or 5 half-lives (whichever is longer) prior to study day 1 that was not reviewed and approved by the principal investigator and the company medical monitor.

History of other malignancy within the past 2 years, with the following exceptions: Malignancy treated with curative intent and with no known active disease present for 2 years before enrollment and felt to be at low risk for recurrence by the treating physician; Adequately treated non-melanoma skin cancer or lentigo maligna without evidence of disease; Adequately treated cervical carcinoma in situ without evidence of disease; Adequately treated breast ductal carcinoma in situ without evidence of disease; Prostatic intraepithelial neoplasia without evidence of prostate cancer; Adequately treated urothelial papillary non-invasive carcinoma or carcinoma in situ.

Previous treatment with a direct KRAS^{G12C} inhibitor.

### Randomization of Subjects

Subjects are randomized in a 1:1 allocation ratio, to either sotorasib 960 mg QD or 240 mg QD, in an open-label manner after meeting all enrollment requirements. The randomization is stratified by number of prior lines of therapy for metastatic disease (1 to 2 or >2), history of CNS metastasis (yes or no), performance status (<2 or 2), and race (Asian vs non-Asian).

### Dose and Administration

Sotorasib is administered orally once daily. No drug holidays are allowed. Subjects should take the sotorasib dose (all tablets at the same time) with or without food at approximately the same time every day. The dose should be taken within a 2 hour window of the scheduled time. A dose of sotorasib can be replaced in the event of vomiting if the vomiting occurs within 15 minutes of the dosing, all tablets administered are accounted for (e.g., 4 tablets must be collected if 4 tablets were administered) and are intact by visual inspection (not broken, partially dissolved, chewed, or crushed). Subjects should skip the sotorasib dose if 6 hours have passed from the scheduled time of dosing.

Dose Interruptions

Subjects randomized to the 960 mg QD arm are allowed up to 2 dose interruptions followed by dose reductions to either 480 mg QD (1 dose lower) or 240 mg QD (2 doses lower), as outlined in Table 8 below. Subjects requiring dose reductions below 240 mg should be permanently discontinued from treatment, as a 240 mg QD dose most readily approximates the exposure profile at lower doses with observed clinical responses. Subjects randomized to the 240 mg QD arm are allowed up to 2 dose interruptions, but sotorasib is not dose reduced upon resuming sotorasib if deemed medically safe and appropriate per the investigator's opinion. Subjects in the 960 mg QD arm who require more than 2 dose reductions due to toxicity management related to sotorasib and subjects in the 240 mg QD arm who require more than 2 dose interruptions due to toxicity management related to sotorasib should be permanently discontinued from treatment.

TABLE 8

| | Recommended Action | |
|---|---|---|
| Toxicity | Hold Until: | Restart Dose:* |
| Grade ≥3 thrombocytopenia | Recovery to grade 1 or less or to | Resume dosing at 1 dose lower |
| | baseline grade (without platelet | |
| | transfusion in last 7 days) | |
| Grade ≥3 febrile neutropenia, or | Recovery to grade 1 or less or to | Resume dosing at 1 dose lower |
| grade ≥3 neutropenia lasting longer | baseline grade | |
| than 7 days | | |
| Grade 4 hemoglobin decrease | Recovery to grade 1 or less or to | Resume dosing at 1 dose lower |
| | baseline grade | |
| Grade ≥3 nausea, vomiting, or | Recovery to grade 1 or less or to | Resume dosing at 1 dose lower |
| diarrhea lasting longer than 3 days | baseline grade | |
| despite optimal medical support | | |
| Suspected interstitial lung disease | ILD/pneumonitis confirmed or excluded | If confirmed, permanently |
| (ILD)/pneumonitis of any grade | | discontinue sotorasib. |
| | | If excluded, restart at same |
| | | dose if no other dose |
| | | modification guidelines are |
| | | applicable. |
| Any other drug-related | Recovery to grade 1 or less or to | Resume dosing at 1 dose lower |
| toxicity ≥grade 3^{a} | baseline grade | |

*For subjects in 240 mg QD arm, subjects are allowed to resume dosing without dose level reductions for toxicity
^{a}For subjects with hepatotoxicity, see below

Hepatotoxicity Guidelines for Sotorasib: Guidelines for management and monitoring of subjects with increased AST, ALT, or alkaline phosphatase (ALP) are presented in Table 9 below.

TABLE 9
If the conditions for permanent discontinuation are met
(below): Participant to be permanently discontinued
AST or ALT >3x ULN and INR >1.5x ULN (for subjects not on
anticoagulation therapy) in the presence of no important
alternative causes for elevated AST/ALT values
OR
AST or ALT >3x ULN and TBL >2x ULN in the presence of no important
alternative causes for elevated AST/ALT and/or TBL values
If conditions are not met: Exclude other causes^{a}
Upon failure to identify any other causes and sotorasib relation to
increase in LFTs cannot be excluded, proceed with guidelines below:

| | Sotorasib | Medical | |
|---|---|---|---|
| CTCAE Grade | Action | Management | Monitoring and Follow-up |
| Grade 2 AST or ALT and | Continue | Consider steroids^{b} | Closely monitor liver function |
| ALP <8x ULN with no clinical | | | tests |
| symptoms consistent with | | | |
| hepatitis | | | |
| (right upper quadrant | | | |
| pain/tenderness, fever, | | | |
| nausea, vomiting, and | | | |
| jaundice) | | | |
| Grade 2 AST or ALT | First | Initiate steroids^{b} | Closely monitor liver function |
| with symptoms | Occurrence | | tests |
| Or | Withhold | | Await resolution to baseline or |
| Grade 3 or 4 | | | grade 1 and resolution or |
| AST or ALT | | | improvement of hepatitis |
| Or | | | symptoms |
| 8x ULN ALP^{d} | | | Restart at 1 dose level |
| | | | reduction^{c, e} |
| | Second | Initiate steroids^{b} | Closely monitor liver function |
| | Occurrence | | tests |
| | Withhold | | Await resolution to baseline or |
| | | | grade 1 and resolution or |
| | | | improvement of hepatitis |
| | | | symptoms |
| | | | Resume at an additional 1 |
| | | | dose level reduction only with |
| | | | MEDICAL MONITOR |
| | | | approval^{c, e} |
| | Third | NOT APPLICABLE | |
| | Occurrence | | |
| | Permanently | | |
| | discontinue | | |
| | Sotorasib | | |

ALP = alkaline phosphatase;
ALT = alanine aminotransferase;
AST = aspartate aminotransferase;
CTCAE = Common Terminology Criteria for Adverse Events;
INR = international normalized ratio;
LFT = liver function test;
TBL = total bilirubin;
ULN = upper limit of normal
^{a}If increase in AST/ALT is likely related to alternative agent, discontinue causative agent and await resolution to baseline or grade 1 prior to resuming sotorasib.
^{b}For example: prednisone 0.25 to 1.0 mg/kg/day or equivalent, followed by a taper.
^{c}Close monitoring at restart (e.g., daily LFTs x 2, then weekly x 4). Sotorasib dose may be increased after discussion with Medical Monitor.
^{d}There is no limit to the number of sotorasib re-challenges for isolated alkaline phosphatase elevations that resolve to baseline or grade 1.
^{e}Dose decrements below 240 mg are not allowable. Subjects may restart at same dose without dose reduction.

Hepatotoxicity Response: Subjects with abnormal hepatic laboratory values (i.e., alkaline phosphatase (ALP), aspartate aminotransferase (AST), alanine aminotransferase (ALT), total bilirubin (TBL)) and/or international normalized ratio (INR) and/or signs/symptoms of hepatitis (as described below) may meet the criteria for withholding or permanent discontinuation of sotorasib.

The following stopping and/or withholding rules apply to subjects for whom another cause of their changes in liver biomarkers (TBL, INR, and transaminases) has not been identified. Important alternative causes for elevated AST/ALT and/or TBL values include, but are not limited to: Hepatobiliary tract disease; Viral hepatitis (e.g., hepatitis A/B/C/D/E, Epstein-Barr Virus, cytomegalovirus, herpes simplex virus, varicella, toxoplasmosis, and parvovirus); Right sided heart failure, hypotension or any cause of hypoxia to the liver causing ischemia; Exposure to hepatotoxic agents/drugs or hepatotoxins, including herbal and dietary supplements, plants and mushrooms; Heritable disorders causing impaired glucuronidation (e.g., Gilbert's syndrome, Crigler-Najjar syndrome) and drugs that inhibit bilirubin glucuronidation (e.g., indinavir, atazanavir); Alpha-one antitrypsin deficiency; Alcoholic hepatitis; Autoimmune hepatitis; Wilson's disease and hemochromatosis; Nonalcoholic fatty liver disease including steatohepatitis; and/or Non-hepatic causes (e.g., rhabdomyolysis, hemolysis).

Rechallenge may be considered if an alternative cause for impaired liver tests (ALT, AST, ALP) and/or elevated TBL, is discovered and/or the laboratory abnormalities resolve to normal or baseline, as described in Table 10 below.

TABLE 10

| Analyte | Temporary Withholding | Permanent Discontinuation |
|---|---|---|
| TBL | >3x ULN at any time | >2x ULN |
| | | OR |
| INR | — | >1.5x (for subjects not on |
| | | anticoagulation therapy) |
| | OR | AND |
| AST/ALT | >5x ULN at any time | In the presence of no important |
| | >3x ULN with clinical signs or symptoms that are | alternative causes for elevated |
| | consistent with hepatitis (such as right upper | AST/ALT and/or TBL values |
| | quadrant pain/tenderness, fever, nausea, | >3x ULN (when baseline was <ULN) |
| | vomiting, and jaundice) | |
| | OR | |
| ALP | >8x ULN at any time | — |

ALP = alkaline phosphatase;
ALT = alanine aminotransferase;
AST = aspartate aminotransferase;
INR = international normalized ratio;
TBL = total bilirubin;
ULN = upper limit of normal

### Radiological Imaging Assessment

The extent of disease is evaluated by contrast-enhanced MRI/CT according to RECIST 1.1, as described below. In order to reduce radiation exposure for subjects, low dose CT should be utilized whenever possible.

The screening scans must be performed within 28 days prior to enrollment and are used as baseline. All subsequent scans are performed in the same manner as at screening, with the same contrast, preferably on the same scanner. Radiological assessment must include MRI/CT of the chest, abdomen and pelvis, as well as assessment of all other known sites of disease. Magnetic resonance imaging (MRI) of the brain should be performed if signs or symptoms suggestive of central nervous system metastases are present.

The same imaging modality, MRI field strength and intravenous and oral contrast agents should be used at screening should be used for all subsequent assessments. Liver specific MRI contrast agents should not be used. To reduce potential safety concerns, macrocyclic gadolinium contrast agents are recommended per National Health Institute guidelines, or follow local standards if more rigorous.

During treatment and follow-up radiological imaging of the chest, abdomen, pelvis, as well as all other known sites of disease, are performed independent of treatment cycle every 6±1 weeks for the first 8 response assessments, with the first postbaseline scan to occur 6±1 weeks after C1D1. After eight 6-week response assessments, radiological imaging and tumor assessment are performed every 12±1 weeks. Radiologic imaging and tumor assessment are performed until disease progression, or end of investigational product, whichever is later. Imaging may also be performed more frequently if clinically necessitated at the discretion of the managing physician. Radiographic response (complete response, partial response) requires confirmation by a repeat scan at least 4 weeks after the first documentation of response and may be delayed until the next scheduled scan to avoid unnecessary procedures. The minimum time interval for determination of stable disease is [symbol] 5 weeks.

All subjects with brain metastasis must have MRI of the brain performed within 28 days prior to first dose of AMG 510. Subsequently, brain scans may be performed at any time if clinically indicated in the judgement of the managing physician. All brain scans on protocol are required to be MRI unless MRI is contraindicated, and then CT with contrast is acceptable.

Radiological imaging assessment during the EOT visit should be performed only for subjects that discontinue treatment for a reason other than disease progression per RECIST 1.1 guidelines.

Determination of disease response for clinical management of subjects is assessed at the clinical sites per RECIST 1.1.

### Independent Central Confirmation of Progression (COP)

When the investigator identifies radiographic progression per RECIST v1.1, the current imaging plus all images to date must be immediately sent to the central imaging vendor. Once any critical queries are resolved, the central imaging vendor performs an independent COP and will provide the study site and Sponsor with a second independent opinion regarding whether the participant has reached progressive disease according to RECIST v1.1. This is performed by a single radiologist that is separate from the central radiologist group reading the images for efficacy. The results of the independent COP are not discussed with the central efficacy reviewers and thus will not influence the determination of response or progression by the central efficacy reviewers. The independent COP is only be utilized to provide a second opinion on the presence or absence of progressive disease according to RECIST v1.1 at the current time point to the site PI and no clinical subject data are to be discussed.

If the evaluation of radiologic disease progression, via central imaging vendor, does not confirm disease progression at this time point, a conference may be organized by the central imaging vendor to be held between the single radiologist and the site radiologist to review the participants' images for determination of confirmation of radiological disease progression. The site PI makes final treatment and subject management decisions.

Progression of radiologic disease should be verified centrally prior to cessation of investigational product, local intervention, initiation of new anti-cancer therapy, or treatment beyond progression. If there are no safety concerns and the study participant is clinically stable, the participant is to remain on investigational product while central confirmation of progression is ongoing and until confirmation of radiologic disease progression is complete.

### Testing for KRAS G12C, PD1, and Various Mutations

The Therascreen® KRAS RGQ PCR Kit from QIAGEN is a real-time qualitative PCR assay performed on the Rotor-Gene Q MDx instrument for the detection of 7 somatic mutations in the human KRAS oncogene using DNA extracted from FFPE tissue. The mutations detected are: G12A, G12D, G12R, G12C, G12S, G12V, G13D. The Therascreen® KRAS RGQ PCR Kit is an investigational in vitro diagnostic device available to be used to test subjects with NSCLC and CRC for the KRAS p.G12C mutation. The Qiagen Therascreen® KRAS RGQ PCR Kit may be approved in certain regions.

PDL1 testing is conducted at the central labs using the Dako PharmDx 22C3 immunohistochemistry FDA-approved kit according to the instructions for use.

### Response Evaluation Criteria in Solid Tumors Version 1.1 (RECIST 1.1)

### Definitions

Measurable Lesions

Measurable Tumor Lesions—Non-nodal lesions with clear borders that can be accurately measured in at least 1 dimension with longest diameter≥10 mm in CT/MRI scan with slice thickness no greater than 5 mm. When slice thickness is greater than 5 mm, the minimum size of measurable lesion should be twice the slice thickness.

Nodal Lesions—Lymph nodes are to be considered pathologically enlarged and measurable, a lymph node must be ≥15 mm in short axis when assessed by CT/MRI (scan slice thickness recommended to be no greater than 5 mm). At baseline and in follow-up, only the short axis is measured and followed. Nodal size is normally reported as two dimensions in the axial plane. The smaller of these measures is the short axis (perpendicular to the longest axis).

Irradiated Lesions—Tumor lesions situated in a previously irradiated area, or in an area subjected to other loco-regional therapy, are not measurable unless there has been demonstrated progression in the lesion prior to enrollment.

Non-measurable Lesions: All other lesions, including small lesions (longest diameter<10 mm or pathological lymph nodes with 10 mm but to <15 mm short axis with CT scan slice thickness no greater than 5 mm) are considered non-measurable and characterized as non-target lesions.

Other examples of non-measurable lesions include: Lesions with prior local treatment: tumor lesions situated in a previously irradiated area, or an area subject to other loco-regional therapy, should not be considered measurable unless there has been demonstrated progression in the lesion; Biopsied lesions; Categorically, clusters of small lesions, bone lesions, inflammatory breast disease, and leptomeningeal disease are non-measurable.

Methods of Measurement

Measurement of Lesions—The longest diameter of selected lesions should be measured in the plane in which the images were acquired (axial plane). All measurements should be taken and recorded in metric notation. All baseline evaluations should be performed as closely as possible to the beginning of treatment and not more than 4 weeks before study Day 1.

Methods of Assessment—The same method of assessment and the same technique should be used to characterize each identified and reported lesion throughout the trial.

CT/MRI—Contrast-enhanced CT or MRI should be used to assess all lesions. Optimal visualization and measurement of metastasis in solid tumors requires consistent administration (dose and rate) of IV contrast as well as timing of scanning. CT and MRI should be performed with 5 mm thick contiguous slices.

Baseline Documentation of “Target” and “Non-Target” Lesions

Target Lesions—All measurable lesions up to a maximum of two (2) lesions per organ and five (5) lesions in total, representative of all involved organs should be identified as target lesions and recorded and measured at baseline.

Target lesions should be selected on the basis of their size (lesions with the longest diameter) and suitability for accurate repeated measurements.

Pathologic lymph nodes (with short axis 15 mm) may be identified as target lesions. All other pathological nodes (those with short axis 10 mm but <15 mm) should be considered non-target lesions.

A sum of the diameters (longest for non-nodal lesions, short axis for nodal lesions) for all target lesions are calculated and reported as the baseline sum of diameters. The baseline sum of diameters are used as reference by which to characterize objective tumor response.

Non-Target Lesions—All other lesions (or sites of disease) including pathological lymph nodes should be identified as non-target lesions and should also be recorded at baseline. Measurements of these lesions are not required, and these lesions should be followed as “present”, “absent”, or “unequivocal progression” throughout the study. In addition, it is possible to record multiple non-target lesions involving the same organ as a single item on the case report form (e.g., “multiple enlarged pelvic lymph nodes” or “multiple liver metastases”).

Response Criteria

TABLE 11
Evaluation of Target Lesions

| | |
|---|---|
| Complete Response (CR): | Disappearance of all target lesions. Any pathological lymph nodes |
| | (whether target or non-target) must have reduction in short axis to <10 mm. |
| Partial Response (PR): | At least a 30% decrease in the sum of the diameters of target lesions, |
| | taking as reference the baseline sum of diameters. |
| Progressive Disease (PD): | At least a relative 20% increase and an absolute increase of 5 mm in |
| | the sum of the diameters of target lesions, taking as reference the |
| | smallest sum on study, or the appearance of 1 or more new lesions. |
| Stable Disease (SD) | Neither sufficient shrinkage to qualify for PR nor sufficient increase to |
| | qualify for PD, taking as reference the smallest sum of diameters |
| | since the treatment started. |

TABLE 12
Evaluation of Non-target Lesions

| | |
|---|---|
| Complete Response (CR): | Disappearance of all non-target lesions and normalization of tumor |
| | marker levels. All lymph nodes must be non-pathological in size (<10 mm short axis). |
| Incomplete Response/Stable | Persistence of one or more non-target lesion(s) or/and maintenance |
| Disease (SD): | of tumor marker levels above the normal limits. |
| Progressive Disease (PD) | Unequivocal progression of existing non-target lesions and/or |
| | appearance of one or more new lesions.¹ |

¹To achieve “unequivocal progression” on the basis of the non-target disease, there must be an overall level of substantial worsening in non-target disease such that, even in presence of SD or PR in target disease, the overall tumor burden has increased sufficiently to merit discontinuation of therapy. A modest “increase” in the size of 1 or more non-target lesions is usually not sufficient to qualify for unequivocal progression status.

Evaluation of Overall Response

The best overall response is the best response recorded from the start of the study treatment until the end of treatment or disease progression/recurrence (taking as reference for PD the smallest measurements recorded since the treatment started).

In general, the subject's best response assignment depends on the findings of both target and non-target disease and also take into consideration the appearance of new lesions.

TABLE 13a
Time Point response: Subjects with Target
(+/− Non-target) Disease

| Target Lesions | Non-target Lesions | New Lesions | Overall Response |
|---|---|---|---|
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or No | PD |
| Any | PD | Yes or No | PD |

NE = Not evaluable

TABLE 13b
Time Point Response: Subjects with Non-Target Disease Only

| Non-Target Lesions | New Lesions | Overall Response |
|---|---|---|
| CR | No | CR |
| Non-CR/non-PD | No | Non-CR/non-PD¹ |
| Not all evaluated | No | NE |
| Unequivocal PD | Yes or No | PD |
| Any | Yes | PD |

¹“Non-CR/non-PD” is preferred over “SD” for non-target disease since SD is increasingly used as endpoint for assessment of efficacy in some trials so as to assign this category when no lesions can be measured is not advised.

TABLE 14
Overall Response: Confirmation of Complete Response
(CR) and Partial Response (PR) required

| Overall Response | Overall Response | |
|---|---|---|
| First Time Point | Second Time Point | Best Overall Response |
| CR | CR | CR |
| CR | PR | SD, PD, or PR¹ |
| CR | SD | SD provided minimum criteria for SD duration met, |
| | | otherwise, PD |
| CR | PD | SD provided minimum criteria for SD duration met, |
| | | otherwise, PD |
| CR | NE | SD provided minimum criteria for SD duration met, |
| | | otherwise, NE |
| PR | CR | PR |
| PR | PR | PR |
| PR | SD | SD |
| PR | PD | SD provided minimum criteria for SD duration met, |
| | | otherwise, PD |
| PR | NE | SD provided minimum criteria for SD duration met, |
| | | otherwise, NE |
| NE | NE | NE |

¹If a CR is truly met at first time point, then any disease at a subsequent time point, even if disease meeting PR criteria relative to baseline, makes the disease PD at that point (since disease must have reappeared after CR). Best response would depend upon whether minimum duration for SD was met. However, sometimes “CR” may be claimed when subsequent scans suggest small lesions were likely still present and in fact the subject had PR, not CR at the first time point. Under these circumstances, the original CR should be changed to PR and the best response is PR.

Special Notes on Response Assessment

Nodal lesions—Lymph nodes identified as target lesions should always have the actual short axis measurement recorded, even if the nodes regress to below 10 mm on study. In order to qualify for CR, each node must achieve a short axis<10 mm, NOT total disappearance. Nodal target lesion short axis measurements are added together with target lesion′ longest diameter measurements to create the sum of target lesion diameters for a particular assessment (time point).

Target lesions that become “too small to measure”—While on study, all lesions (nodal and non-nodal) recorded at baseline should have their measurements recorded at each subsequent evaluation. If a lesion becomes less than 5 mm, the accuracy of the measurement becomes reduced. Therefore, lesions less than 5 mm are considered as being “too small to measure”, and are not measured. With this designation, they are assigned a default measurement of 5 mm. No lesion measurement less than 5 mm should be recorded, unless a lesion totally disappears and “0” can be recorded for the measurement.

New lesions—The term “new lesion” always refers to the presence of a new finding that is definitely tumor. New findings that only may be tumor, but may be benign (infection, inflammation, etc.) are not selected as new lesions, until that time when the review is certain they represent tumor.

If a new lesion is equivocal, for example because of its small size, continued therapy and follow-up evaluation will clarify if it represents truly new disease. If repeat scans confirm there is definitely a new lesion, then progression should be declared using the date of the initial scan.

A lesion identified on a follow-up study in an anatomical location that was not scanned at baseline is considered a new lesion and will indicate disease progression, regardless of any response that may be seen in target or non-target lesions present from baseline.

Subjects with a global deterioration of health status requiring discontinuation of treatment without objective evidence of disease progression at that time should be classified as having “symptomatic deterioration.” Every effort should be made to document the objective progression with an additional imaging assessment even after discontinuation of treatment.

In some circumstances it may be difficult to distinguish residual disease from scar or normal tissue. When the evaluation of complete response (CR) depends on this determination, it is recommended that the residual lesion be further investigated by fluorodeoxyglucose-positron emission tomography (FDG-PET) or PET/computed tomography (PET/CT), or possibly fine needle aspirate/biopsy, to confirm the CR status.

Confirmation Measurement/Duration of Response

Response Confirmation—In non-randomized trials where response is the primary endpoint, confirmation of PR and CR is required to ensure responses identified are not the result of measurement error.

Duration of overall response—The duration of overall response is measured from the time measurement criteria are first met for CR/PR (whichever is first recorded) until the first date the recurrent or progressive disease is objectively documented or death, whichever is earlier.

Duration of Stable Disease—SD is measured from the start of the treatment until the criteria for disease progression are met, taking as reference the smallest measurements recorded since the treatment started, or death, whichever is earlier.

Preliminary data (Feb. 21, 2022):

Provided below is a table summarizing the pharmacokinetic (PK) data following administration of sotorasib (240 mg or 960 mg) on day 1 and day 8.

TABLE 15

| | | | Tₘₐₓ | Cₘₐₓ | AUC₀₋₂₄ₕ | |
|---|---|---|---|---|---|---|
| Dose | Day | N | (hour) | (ug/mL) | (hr*ug/ml | T_{1/2,z} (hr) |
| 240 mg | 1 | 35 | 1.0 | 5.93 | 57.1 | 5.74 |
| | | | *1.0-24) | (7.78, 71%) | (81.6, 102%)^{a} | (5.90, 24%)^{b} |
| | 8 | 29 | 4.0 | 4.76 | 36.3 | 5.07 |
| | | | (1.0-4.0) | (5.68, 63%) | (40.7, 53%)^{c} | (5.20, 22%)^{d} |
| 960 mg | 1 | 37 | 4.0 | 8.28 | 85 | 5.59 |
| | | | (1.0-4.0) | (10.2, 60%) | (107, 70%)^{e} | (5.66, 27%)^{f} |
| | 8 | 30 | 4.0 | 5.54 | 50.3 | 4.67 |
| | | | (1.0-4.0 | (6.95, 68%) | (62.6, 65%)^{g} | (4.75, 18%)^{h} |

^{a}N = 33;
^{b}N = 19;
^{c}N = 27;
^{d}N = 18;
^{e}N = 36;
^{f}N = 15;
^{g}N = 29;
^{h}N = 16;
Data presented as GeoMean (Mean, CV %) for all PK Parameters except for tₘₐₓ, which is presented as Median (Range). Values are reported to 3 significant figures except for tₘₐₓ and CV % which are presented as two significant figures and the nearest integer, respectively.
tₘₐₓ = Time to reach Cₘₐₓ;
Cₘₐₓ = Maximum observed drug concentration;
AUC₀₋₂₄ₕ = area under the concentration-time curve from time 0 to 24 hr postdose;
t_{1/2,z} = half-life

Briefly, numerically higher mean exposures (Cₘₐₓ and AUC₀₋₂₄ₕ) were observed on Day 1 and Day 8 following 960 mg compared to 240 mg. The 960 mg PK was consistent with anticipated exposure ranges and elimination half-life. Exposures on Day 8 were 30-40% lower than on Day 1 for both the 240 mg and 960 dosing, which is consistent with anticipated steady-state profile.

Further, preliminary data of two NSCLC adenocarcinoma (stage IV) patients treated with 240 mg sotorasib once daily was reviewed. One patient, after 9 cycles on treatment with 240 mg sotorasib, exhibited stable disease (SD) after cycle 3 and 5, and partial response (PR) after cycles 7 and 9. One patient, after 9 cycles on treatment, exhibited partial response (PR) after cycles 3, 5, and 7, and after cycle 9 exhibited progression disease (PD) with new lesions.

### Example 4— Sotorasib Dose Reduction Protocol

The most common adverse events to sotorasib treatment during a phase 2 Study included laboratory abnormalities (≥10%), decreased lymphocytes, decreased hemoglobin, diarrhea, musculoskeletal pain, increased aspartate aminotransferase, increased alanine aminotransferase, decreased calcium, increased alkaline phosphatase, increased urine protein, decreased sodium, nausea, fatigue, decreased albumin, increased activated partial thromboplastin time, cough vomiting, constipation, dyspnea and abdominal pain.

TABLE 16
Common adverse events during Phase 2 Study

| | | |
|---|---|---|
| ADVERSE event | ALL GRADES (%) | GRADE 3 TO 4 (%) |

Gastrointestinal disorders

| | | |
|---|---|---|
| Diarrhea | 42 | 5 |
| Nausea | 26 | 1.0 |
| Vomiting | 17 | 1.5 |
| Constipation | 16 | 0.5 |
| Abdominal pain* | 15 | 1.0 |

Hepatobiliary disorders

| | | |
|---|---|---|
| Hepatotoxicity | 25 | 12 |

Respiratory

| | | |
|---|---|---|
| Cough | 20 | 1.5 |
| Dyspnea | 16 | 2.9 |

Musculoskeletal and connective tissue disorders

| | | |
|---|---|---|
| Musculoskeletal pain | 35 | 2.0 |
| Arthalgia | 12 | 1.0 |

General disorders and administration site conditions

| | | |
|---|---|---|
| Fatigue | 25 | 2.0 |

Metabolism and nutrition disorders

| | | |
|---|---|---|
| Decreased appetite | 13 | 1.0 |

Infections and infestations

| | | |
|---|---|---|
| Pneumonia | 12 | 7 |

Skin and subcutaneous tissue disorders

| | | |
|---|---|---|
| Rash | 12 | 0 |

Nervous system disorders

| | | |
|---|---|---|
| Headache | 10 | 0 |

*Grading defined by NCI CTCAE v. 5.0; Abdominal pain includes both upper and lower abdominal pain; Hepatotoxicity includes: increased alanine aminotransferase, increased aspartate aminotransferase, increased blood bilirubin, drug-induced liver injury, hepatitis, abnormal transaminases, and increased transaminases

A dose reduction (from a total daily dose of 960 mg to 480 mg, or from 480 mg to 240 mg) was permitted when certain adverse events (e.g., hepatotoxicity, nausea/vomiting, diarrhea, other adverse reactions) were observed. The dose reduction levels are summarized below in Table 17.

TABLE 17

| Dose reduction level | Dose |
|---|---|
| First dose reduction | 480 mg daily |
| Second dose reduction | 240 mg daily |

TABLE 18
Adverse reactions and associated recommended actions

| | | RECOMMENDED ACTION | |
|---|---|---|---|
| ADVERSE EVENT | SEVERITY | HOLD UNTIL: | RESTART DOSE:* |
| Hepatotoxicity | Grade 2 AST or ALT with symptoms | Recovery to grade 1 or less or | Resume dosing at |
| | Or | to baseline grade | next lower dose |
| | Grade 3 to 4 AST or ALT | | level |
| | AST or ALT >3 x ULN with total | Permanently discontinue | N/A |
| | bilirubin >2 x ULN in the absence of | treatment | |
| | alternative causes | | |
| Interstitial lung disease | Any grade | Withhold treatment if | N/A |
| (ILD)/pneumonitis | | ILD/pneumonitis is suspected | |
| | | Permanently discontinue | |
| | | treatment is ILD/pneumonitis is | |
| | | confirmed | |
| Nausea or vomiting | Grade 3 to 4 | Recovery to grade 1 or less or | Resume dosing at |
| despite appropriate | | to baseline grade | next lower dose |
| supportive care | | | level |
| (including anti-emetic | | | |
| therapy) | | | |
| Diarrhea despite | Grade 3 to 4 | Recovery to grade 1 or less or | Resume dosing at |
| appropriate supportive | | to baseline grade | next lower dose |
| care (including anti- | | | level |
| diarrheal therapy) | | | |
| Other adverse reactions | Grade 3 to 4 | Recovery to grade 1 or less or | Resume dosing at |
| | | to baseline grade | 1 dose lower |

Out of the 427 subjects that received sotorasib monotherapy with any tumor type and at any dose, 56 subjects (13.1%) had a dose reduction, with most primary reason being due to adverse events (AEs) (46 subjects, 10.8%). See Table 19 below.

TABLE 19
Summary of sotorasib dose reduction (safety analysis set)

| | Any tumor | Any tumor | Any tumor | Any tumor | Any tumor | Any tumor | Total any |
|---|---|---|---|---|---|---|---|
| | types 180 | types 360 | types 720 | types 960 | types 480 | types 960 | tumor types |
| | mg QD | mg QD | mg QD | mg QD | mg BID | mg QD | and any |
| | fasted | fasted | fasted | fasted | fed | fed | dose |
| | (n = 6) | (n = 27) | (n = 11) | (n = 339) | (n = 26) | (n = 18) | (n = 427) |
| Number of subjects | 0 (0.0) | 3 (11.1) | 2 (18.2) | 36 (13.6) | 3 (11.5) | 2 (11.1) | 56 (13.1) |
| with any dose | | | | | | | |
| reduction-n (%) | | | | | | | |
| Primary reason(s) | | | | | | | |
| for dose reduction | | | | | | | |
| Adverse event | 0 (0.0) | 3 (11.1) | 1 (9.1) | 38 (11.2) | 2 (7.7) | 2 (11.1) | 46 (10.8) |
| Noncompliance | 0 (0.0) | 1 (3.7) | 0 (0.0) | 1 (0.3) | 0 (0.0) | 0 (0.0) | 2 (0.5) |
| Dose | 0 (0.0) | 0 (0.0) | 0 (0.0) | 3 (0.9) | 1 (3.8) | 0 (0.0) | 4 (0.9) |
| administration error | | | | | | | |
| Per protocol | 0 (0.0) | 0 (0.0) | 0 (0.0) | 1 (0.3) | 0 (0.0) | 0 (0.0) | 1 (0.2) |
| Weight change | 0 (0.0) | 0 (0.0) | 0 (0.0) | 0 (0.0) | 0 (0.0) | 0 (0.0) | 0 (0.0) |
| PI decision | 0 (0.0) | 0 (0.0) | 0 (0.0) | 3 (0.9) | 0 (0.0) | 0 (0.0) | 3 (0.7) |
| Other | 0 (0.0) | 0 (0.0) | 1 (9.1) | 10 (2.9) | 0 (0.0) | 0 (0.0) | 11 (2.6) |

Out of the 56 subjects who had a dose reduction, 39 subjects (69.6%) discontinued treatment with sotorasib (Table 20) and the remaining 17 subjects continued with treatment as of the data cut-off date.

TABLE 20
Disposition and discontinuation reason for subjects with any dose reduction.

| | Any tumor | Any tumor | Any tumor | Any tumor | Any tumor | Any tumor | Total any |
|---|---|---|---|---|---|---|---|
| | types 180 | types 360 | types 720 | types 960 | types 480 | types 960 | tumor types |
| | mg QD | mg QD | mg QD | mg QD | mg BID | mg QD | and any |
| | fasted | fasted | fasted | fasted | fed | fed | dose |
| | (N = 0) | (N = 3) | (N = 2) | (N = 46) | (N = 3) | (N = 2) | (N = 56) |
| | n (%) | n (%) | n (%) | n (%) | n (%) | n (%) | n (%) |
| Subjects who | 0 (−) | 2 (66.7) | 2 (100.0) | 32 (69.6) | 2 (66.7) | 1 (50.0 | 39 (69.6) |
| discontinued sotorasib | | | | | | | |
| Adverse event | 0 (0.0) | 0 (0.0) | 0 (0.0) | 9 (19.6) | 2 (66.7) | 1 (50.0) | 12 (21.4) |
| Decision by sponsor | 0 (0.0) | 0 (0.0) | 0 (0.0) | 0 (0.0) | 0 (0.0) | 0 (0.0) | 0 (0.0) |
| Death | 0 (0.0) | 0 (0.0) | 0 (0.0) | 0 (0.0) | 0 (0.0) | 0 (0.0) | 0 (0.0) |
| Subject request | 0 (0.0) | 0 (0.0) | 0 (0.0) | 0 (0.0) | 0 (0.0) | 0 (0.0) | 1 (1.8) |
| Pregnancy | 0 (0.0) | 0 (0.0) | 0 (0.0) | 0 (0.0) | 0 (0.0) | 0 (0.0) | 1 (1.8) |
| Non-Compliance | 0 (0.0) | 0 (0.0) | 0 (0.0) | 0 (0.0) | 0 (0.0) | 0 (0.0) | 0 (0.0) |
| Disease progression | 0 (0.0) | 2 (66.7) | 2 (100.0) | 21 (45.7) | 0 (0.0) | 0 (0.0) | 25 (44.6) |
| Requirement for | 0 (0.0) | 0 (0.0) | 0 (0.0) | 0 (0.0) | 0 (0.0) | 0 (0.0) | 0 (0.0) |
| alternative therapy | | | | | | | |
| Protocol specified | 0 (0.0) | 0 (0.0) | 0 (0.0) | 0 (0.0) | 0 (0.0) | 0 (0.0) | 0 (0.0) |
| criteria | | | | | | | |

Of the 39 subjects who discontinued sotorasib treatment, there were only 12 subjects (21.4%) whose reason to discontinue was due to AEs, confirming that most subjects that received a modified dosed of sotorasib did not require permanent discontinuation of sotorasib treatment due to an AE. The majority of the 39 subjects who discontinued sotorasib after dose reduction did so due to progression of disease (25 subjects, 44.6%). The data suggests that, unexpectedly considering the non-linear pharmacokinetic properties of sotorasib as shown herein, dose reduction improved the individual safety profile and may have continued to the low rate of discontinuing sotorasib due to AEs.

### Example 5— Contraindication with Co-Administration of Sotorasib with Acid-Reducing Agents Under Fasted Conditions

This Phase 1, open-label, fixed-sequence study enrolled 14 healthy subjects. Subjects received 960 mg sotorasib on Day 1, 40 mg omeprazole once daily on Days 4 to 8, and 40 mg omeprazole followed by 960 mg sotorasib on Day 9. All doses were administered under fasted conditions. Blood samples for sotorasib PK were collected predose and up to 48 hours post-sotorasib dose. Sotorasib plasma PK parameters were estimated using non-compartmental methods.

Coadministration of sotorasib with omeprazole delayed sotorasib time to maximal plasma concentration (Lx) by 0.75 hours. Mean terminal half-life (ty2) of sotorasib was similar following coadministration of sotorasib with omeprazole compared to administration of sotorasib alone. Geometric mean sotorasib AUC_{inf} (area under the curve from time zero to infinity) and Cₘₐₓ (maximal plasma concentration) following coadministration of sotorasib with omeprazole (17000 h*ng/mL and 3100 ng/mL, respectively) were lower compared to administration of sotorasib alone (29300 h*ng/mL and 7200 ng/mL, respectively). Sotorasib was safe and well tolerated when coadministered with 40 mg omeprazole or administered alone to healthy subjects.

Results indicated that coadministration of sotorasib with omeprazole, in the fasted state, decreased sotorasib AUC_{inf} by 42% and Cₘₐₓ by 57% compared with administration of sotorasib alone.

### Example 6— Contraindication with Co-Administration of Sotorasib with Acid-Reducing Agents Under Fed Conditions

This was a phase 1, open-label, fixed sequence, crossover, single-center study to explore mitigation strategies to limit the impact of acid-reducing agents on the exposure of sotorasib. This study evaluated the PK of sotorasib administered alone and in combination with famotidine or omeprazole in healthy men and women (a total of 14 subjects) under fed conditions. Subjects received a single dose of sotorasib on day 1, an evening dose of famotidine on day 3 (10 hours prior to sotorasib administration), a single dose of sotorasib on day 4 followed by another dose of famotidine 2 hours later, daily doses of omeprazole on day 6 through day 10, and a single dose of both omeprazole and sotorasib on day 11. All sotorasib administrations occurred following consumption of a standard calorie moderate fat meal. Blood was collected at predetermined timepoints to characterize plasma concentrations of sotorasib. Safety and tolerability monitoring was performed throughout the study.

A total of 15 healthy subjects (1 woman and 13 men) were enrolled in the study. Thirteen out of the 14 subjects received all treatments and completed the study.

Geometric least-square mean ratios of sotorasib AUC_{inf} and Cₘₐₓ were 0.622 and 0.654, respectively when comparing sotorasib coadministered with famotidine and sotorasib alone under fed conditions. Geometric least-square mean ratios of sotorasib AUC_{inf} and Cₘₐₓ were 0.430 and 0.349, respectively, when comparing sotorasib coadministered with omeprazole and sotorasib alone. Doses of 960 mg sotorasib were safe and well tolerated with coadmnistered with a single dose of 40 mg famotidine and following multiple daily dosing of 40 mg omeprazole under fed conditions to healthy subjects.

In summary, coadministration of a single dose of famotidine (H2 receptor antagonist) given 10 hours prior to and 2 hours after a single dose of sotorasib under fed conditions decreased sotorasib Cₘₐₓ by 35% and AUC by 38%. In addition, co-administration of repeat doses of omeprazole (PPI) with a single dose of sotorasib decreased sotorasib Cₘₐₓ by 65% and AUC by 57% under fed conditions.

### Example 7—Contraindication with Coadministration of Sotorasib with Strong CYP34A4 Inducers

This Phase 1, open-label, fixed-sequence study enrolled 14 healthy subjects. Each subject received 960 mg sotorasib on Days 1, 3 and 18, and 600 mg rifampin on Day 3 and Days 5 to 19. Blood samples for sotorasib PK were collected predose and up to 48 hours post-sotorasib dose. Sotorasib plasma PK parameters were estimated using non-compartmental methods.

Results:

Geometric mean sotorasib AUC_{inf} (area under the curve from time zero to infinity) and Cₘₐₓ (maximal plasma concentration) following coadministration of single dose of rifampin with sotorasib (19600 h*ng/mL and 5340 ng/mL, respectively), were similar to those of sotorasib alone (25600 h*ng/mL and 6350 ng/mL, respectively). Geometric mean sotorasib AUO_{nf} and Cₘₐₓ following coadministration of multiple doses of rifampin with sotorasib (12400 h*ng/mL and 4110 ng/mL, respectively), were lower compared to those of sotorasib alone (25600 h*ng/mL and 6350 ng/mL, respectively).

Sotorasib was safe and well tolerated when coadministered with 600 mg rifampin or administered alone to healthy subjects. Single dose of rifampin did not have a clinically meaningful effect on sotorasib PK indicating sotorasib is not a substrate of OATP1B1. Multiple doses of rifampin decreased sotorasib AUO_{nf} by 51% and Cₘₐₓ by 35%, indicating sotorasib is a CYP3A4 substrate, consistent with in vitro data.

### Example 8—Contraindication with Coadministration of Sotorasib with CYP34A Substrates

This Phase 1, open-label, fixed-sequence study enrolled 5 subjects with previously untreated NSCLC who received a single, oral dose of 2 mg midazolam alone of day −1, 960 mg sotorasib orally on days 1 through 14, and a single oral dose of 2 mg midazolam at approximately the same time as an oral dose of 960 mg sotorasib on day 15. Blood samples for sotorasib PK were collected predose and up to 48 hours post-sotorasib dose. Sotorasib plasma PK parameters were estimated using non-compartmental methods.

Single dose plasma midazolam PK data were obtained from 5 subjects who received midazolam alone and midazolam coadministered with sotorasib following 14 days of multiple daily dosing of sotorasib. Results indicated that exposure to midazolam decreased when coadministered with sotorasib following multiple daily dosing with sotorasib. Coadministration of sotorasib with midazolam (a sensitive CYP3A4 substrate) decreased midazolam Cₘₐₓ by 48% and AUV by 53%.

### Example 9—Contraindication with Coadministration of Sotorasib and P-Gp Substrates

This Phase 1, open-label, fixed-sequence study enrolled 14 healthy subjects. Each subject received 0.5 mg digoxin on Day 1 and 960 mg sotorasib followed by 0.5 mg digoxin on Day 7. Blood samples for digoxin PK were collected predose and up to 144 hours post-digoxin dose. Samples were measured using validated high-performance liquid chromatography tandem mass spectrometry methods. PK parameters were estimated using non-compartmental methods. Safety and tolerability were monitored throughout the study.

Digoxin median time to maximal plasma concentration (tₘₐₓ) and mean terminal half-life (t_{1/2}) were similar following coadministration of digoxin with sotorasib compared to those of digoxin alone. Geometric mean digoxin AUC_{inf} (area under the curve from time zero to infinity) following coadministration of digoxin with sotorasib (40.3 h*ng/mL) was similar to that of digoxin alone (33.2 h*ng/mL). Geometric mean digoxin Cₘₐₓ (maximal plasma concentration) following coadministration of digoxin with sotorasib (3.64 ng/mL) was higher compared to that of digoxin alone (1.90 ng/mL). Single doses of 0.5 mg digoxin were safe and well tolerated when administered alone or coadministered with 960 mg sotorasib.

Results indicated that coadministration of digoxin with a single dose of sotorasib increased digoxin AUC_{inf} and Cₘₐₓ by approximately 21% and 91%, respectively, compared with digoxin alone.

### REFERENCES

Albert et al. 2007 Nat. Methods 4:903-905
Alizadeh et al. 1996 Nat. Genet. 14:457-460
Beers and Nederlof, 2006 Breast Cancer Res. 8(3):210
Bertone et al. 2006 Genome Res 16(2):271-281
Canon, et al. Nature 2019, 575(7781), 217.
Cerami, et al. Cancer Discov. 2012, 2(5), 401.
Chung et al. 2004 Genome Res. 14(1):188-196
Cully M, Downward J. SnapShot: Ras Signaling. Cell. 2008; 133:1292.
Dalma-Weiszhausz et al. 2006 Methods Enzymol. 410:3-28
Eisenhauer, et al., Eur. J. Cancer, 2009 45:228-247
Forshew et al., 2012 Sci Transl Med; 4:136ra68
Gao, et al. Science Signaling 2013, 6(269), pl1.
Haber and Velculescu, 2014 Cancer Discov., 4:650-61
Hong, et al.. N. Engl. J. Med. 2020, 383, 1207.
Hughes et al. 2001 Nat. Biotechnol. 19(4):342-347
Irizarry 2003 Nucleic Acids Res 31:e15
Janes et al. Cell. 2018; 172(3):578-589.
Jasmine et al. 2012 PLoS One 7(2):e31968
Kim et al. 2006 Carcinogenesis 27(3):392-404
Kinde et al., 2011 Proc Natl Acad Sci USA; 108:9530-5
Kumar et al. 2012 J. Pharm. Bioallied Sci. 4(1):21-26
Laere et al. 2009 Methods Mol. Biol. 512:71-98
Lanman, et al. J. Med. Chem. 2020, 63, 52.
Lin et al. 2010 BMC Genomics 11:712
Liu et al. 2017 Biosens Bioelectron 92:596-601
Lodes et al. 2009 PLoS One 4(7):e6229
Mackay et al. 2003 Oncogene 22:2680-2688
Mao et al. 2007 Curr. Genomics 8(4):219-228
McDonald et al. Cell. 2017; 170(3):577-592.
Michels et al. 2007 Genet. Med. 9:574-584
Mockler and Ecker, 2005 Genomics 85(1):1-15
Ostrem, et al. Nature. 2013; 503:548-551.
Ostrem and Shokat. Nature Rev Drug Discov. 2016; 15(11):771-785.
Patricelli et al. Cancer Discovery. 2016; 6:316-329.
Pinkel et al. 2005 Nat. Genetics 37:S11-S17
Simanshu et al. Cell. 2017; 170:17-33.
Thomas et al. 2005 Genome Res. 15(12):1831-1837
Thompson et al., 2012 PLoS ONE, 7:e31597
Wang et al. 2012 Cancer Genet 205(7-8):341-55
Wei et al. 2008 Nucleic Acids Res 36(9):2926-2938
Xie et al. Front Pharmacol. 2017; 8:823.
Zubrod et al., J Chronic Disease, 1960 11:7-33

1. A method of treating cancer in a patient comprising administering a total daily dose of 240 mg sotorasib to the patient, wherein the cancer is a KRAS G12C mutated cancer.

2. A method of treating cancer in a patient comprising administering an initial total daily dose of 960 mg sotorasib to the patient, and administering a reduced total daily dose of sotorasib of 480 mg when the patient experiences an adverse event to the initial total daily dose, wherein the cancer is a KRAS G12C mutated cancer.

3. The method of claim 2, further comprising administering a second reduced total daily dose of sotorasib of 240 mg when the patient experiences an adverse event to the reduced total daily dose.

4. The method of claim 2 or claim 3, wherein the adverse event is an elevation of one or more liver enzymes in the patient, wherein the liver enzyme is alanine aminotransferase (ALT) or aspartate aminotransferase (AST).

5. The method of claim 4, wherein the elevated level of ALT and/or AST is >3×ULN if baseline was normal, >3.0× baseline if baseline was abnormal.

6. The method of claim 5, further comprising withholding sotorasib treatment from the patient until ALT and/or AST levels in the patient improve to Grade 1 or less or to baseline before administering the reduced total daily dose of sotorasib or the second reduced total daily dose of sotorasib.

7. The method of claim 4, comprising discontinuing sotorasib treatment when levels of AST or ALT>3×ULN with total bilirubin>2×ULN in the absence of alternative causes.

8. The method of claim 2 or claim 3, wherein the adverse event is diarrhea, wherein the diarrhea is of Grade 3 or Grade 4.

9. The method of claim 8, further comprising withholding sotorasib treatment from the patient until diarrhea in the patient improves to Grade 1 or less or to baseline before administering the reduced total daily dose of sotorasib or the second reduced total daily dose of sotorasib.

10. The method of claim 2 or claim 3, wherein the adverse event is nausea or vomiting, wherein the nausea or vomiting is of Grade 3 or Grade 4.

11. The method of claim 10, further comprising withholding sotorasib treatment from the patient until the nausea or vomiting in the patient improves to Grade 1 or to baseline before administering the reduced total daily dose of sotorasib or the second reduced total daily dose of sotorasib.

12. The method of claim 2 or claim 3, wherein the sotorasib is administered once per day.

13. The method of claim 2 or claim 3, wherein the sotorasib is administered orally.

14. (canceled)

15. The method of claim 2 or claim 3, wherein the cancer is non-small cell lung cancer.

16. The method of claim 15, wherein the cancer is locally advanced or metastatic non-small cell lung cancer.

17. (canceled)

18. The method of claim 2 or claim 3, wherein the cancer is colorectal cancer.

19. The method of claim 2 or claim 3, wherein the cancer is pancreatic cancer.

20. The method of claim 2 or claim 3, wherein the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma.

21.-64. (canceled)
